(19) United States
(12) Patent Application Publication
(10) Pub. No.: US 2008/0292690 A1
(43) Pub. Date: Nov. 27, 2008

(54) MULTI-MEMBRANE IMMUNOISOLATION SYSTEM FOR CELLULAR TRANSPLANT

(75) Inventor: Taylor G. Wang, Nashville, TN (US)

Correspondence Address: EDELL, SHAPIRO & FINNAN, LLC, 1901 RESEARCH BOULEVARD, SUITE 400, ROCKVILLE, MD, 20850, US

(73) Assignee: Technology Center

(21) Appl. No.: 11/870,366

(22) Filed: Oct. 10, 2007

Related U.S. Application Data
(63) Continuation-in-part of application No. 11/399,390, filed on Apr. 7, 2006.

(51) Int. Cl. A61K 9/48; A61K 35/12; A61P 5/50

(52) U.S. Cl. 424/451; 424/93.7

(57) ABSTRACT

This invention relates to an immunoisolation encapsulation system that protects cellular transplants and thus allows cell function and survival without the need of immunosuppression. The immunoisolation system is a multi-component, multi-membrane capsule that allows optimization of multiple design parameters independently for reproducible functions in large animals models.

### RELATED APPLICATIONS

This application claims benefit of priority as a continuation-in-part of U.S. patent application Ser. No. 11/399,390, filed Apr. 7, 2006.

### FEDERAL FUNDING LEGEND

This invention was produced in part using funds from the Federal government under NASA contract NAG 5-12429. Accordingly, the Federal government has certain rights in this invention.

### FIELD OF THE INVENTION

his invention relates to a multi-membrane immunoisolation system for cellular transplant that can be used in large animals and humans without immunosuppression.

### BACKGROUND OF THE INVENTION

The World Health Organization estimates that, as of the year 2000, over 176 million people suffer from diabetes mellitus worldwide. It is predicted that this number will more than double by the year 2030. In patients with insulin-dependent or type 1 diabetes mellitus, autoimmune processes destroy the insulin-producing beta cells of the pancreatic islets. Injection of human insulin, while somewhat effective, does not precisely restore normal glucose homeostasis, which can lead to serious complications such as diabetic nephropathy, retinopathy, neuropathy and cardiovascular disease.

Recently, cellular transplantation has generated enthusiasm for treating a number of human diseases characterized by hormone or protein deficiencies, such as diabetes, Parkinson disease, Huntington disease, and others. However, a number of technical and logistical challenges have prevented cellular transplantation from working effectively. In particular, transplanted cells must be protected from immune attack by the transplant recipient. This often requires potent immunosuppressive agents having considerable toxicity that can expose the patient to a wide variety of serious side effects.

An alternative approach is to enclose the transplanted cells within a semi-permeable membrane. In theory, the semi-permeable membrane is designed to protect cells from immune attack while allowing for both the influx of molecules important for cell function/survival and the efflux of the desired cellular product. This immunoisolation approach has two major potentials: i) cell transplantation without the need for immunosuppressive drugs and their accompanying side effects, and ii) use of cells from a variety of sources such as autografts (host stem-cell derived), allografts (either primary cells or stem-cell derived), xenografts (porcine cells or others), or genetically engineered cells. While this technique has been effective in treating small mammals, such as rodents, the techniques were found to be ineffective when used to treat larger mammals.

Certain immunoisolation systems have been tested in large animal models, but many of those experiments were performed on spontaneous diabetic subjects or utilized immunosuppressive agents. See Sun et al. “Normalization of diabetes in spontaneously diabetic cynomolgus monkeys by xenografts of microencapsulated porcine islets without immunosuppressant,” J. Clin. Invest. 98:1417-22 (1996); Lanza et al., “Transplantation of islets using microencapsulation: studies in diabetic rodents and dogs,” J. Mol. Med. 77(1): 206-10 (1999); Calafiore R., “Transplantation of minimal volume microcapsules in diabetic high mammalians,” Ann NY Acad. Sci. 875: 219-32 (1999); Hering et al., “Long term (>100 days) diabetes reversal in immunosuppressed nonhuman primate recipients of porcine islet xenographs,” American J. Transplantation, 4: 160-61 (2004); and Soon-Shiong et al., “Insulin independence in a Type 1 diabetic patient after encapsulated islet transplantation,” Lancet 343:950-951 (1994). Moreover, many of these experiments could not be reproduced to acceptable scientific standards. The lack of experimental control and consistency of those experiments has complicated scientific interpretation and limited their applicability.

Clearly, the promise of immunoprotection of living cells to treat hormone-deficient diseases has not been realized. Accordingly, what is needed in the art is a reproducible and effective cell therapy treatment that can be used in large mammals without the use of immunosuppressive drugs. This invention answers that need.

### BRIEF SUMMARY OF THE INVENTION

This invention relates to a multi-membrane composition for encapsulating biological material, comprising (a) an inner membrane that is biocompatible with the biological material and possesses sufficient mechanical strength to hold the biological material within the membrane and provide immunoprotection from antibodies in the immune system of a host; (b) a middle membrane that possesses sufficient chemical stability to reinforce the inner membrane from the chemicals in the host; and (c) an outer membrane that is biocompatible with the host and possesses sufficient mechanical strength to shield the inner and middle membranes from non-specific immune response systems in the immune system of the host. The middle membrane also binds the inner membrane with the outer membrane.

This invention also relates to a multi-membrane composition capable of encapsulating biological material, that includes (a) a membrane comprising sodium alginate (SA), cellulose sulfate (CS), poly(methylene-co-guanidine) (PMCG), and calcium chloride (CaCl₂); (b) a membrane comprising a polycation; and (c) a membrane comprising a carbohydrate polymer having carboxylate or sulfate groups. The polycation is a poly-L-lysine, poly-D-lysine, poly-L,D-lysine, polyethylenimine, polyallylamine, poly-L-ornithine, poly-D-ornithine, poly-L,D-ornithine, poly-L-aspartic acid, poly-D-aspartic acid, poly-L,D-aspartic acid, polyacrylic acid, poly-L-glutamic acid, poly-D-glutamic acid, poly-L,D-glutamic acid, succinylated poly-L-lysine, succinylated poly-D-lysine, succinylated poly-L,D-lysine, chitosan, polyacrylamide, poly(vinyl alcohol) or a combination thereof.

This invention also relates to a multi-membrane composition capable of encapsulating biological material wherein the composition is a capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%. The capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams.

This invention also relates to a method of treating a subject suffering from diabetes or related disorders, comprising administering to the subject sufficient amounts of a composition containing insulin-producing islet cells, wherein the composition is a multi-membrane capsule that includes (a) an inner membrane that is biocompatible with the biological material and possesses sufficient mechanical strength to hold the biological material within the membrane and provide immunoprotection from antibodies in the immune system of the subject; (b) a middle membrane that possesses sufficient chemical stability to reinforce the inner membrane from the chemicals in the subject; and (c) an outer membrane that is biocompatible with the host and possesses sufficient mechanical strength to shield the inner and middle membranes from non-specific immune response systems in the immune system of the subject.

The invention also relates to a method of treating a subject suffering from diabetes or related disorders, comprising administering to the subject sufficient amounts of a composition containing insulin-producing islet cells, wherein the composition is a multi-membrane capsule that includes (a) a membrane comprising sodium alginate (SA), cellulose sulfate (CS), poly(methylene-co-guanidine) (PMCG), and calcium chloride (CaCl₂); (b) a membrane comprising a polycation; and (c) a membrane comprising a carbohydrate polymer having carboxylate or sulfate groups. The polycation is poly-L-lysine, poly-D-lysine, poly-L,D-lysine, polyethylenimine, polyallylamine, poly-L-omithine, poly-D-omithine, poly-L,D-omithine, poly-L-aspartic acid, poly-D-aspartic acid, poly-L,D-aspartic acid, polyacrylic acid, poly-L-glutamic acid, poly-D-glutamic acid, poly-L,D-glutamic acid, succinylated poly-L-lysine, succinylated poly-D-lysine, succinylated poly-L,D-lysine, chitosan, polyacrylamide, poly(vinyl alcohol), or a combination thereof.

This invention also relates to a method of treating a subject suffering from diabetes or related disorders, comprising administering to the subject sufficient amounts of a composition containing insulin-producing islet cells, wherein the composition is a multi-membrane capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%. The capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams. This invention further relates to an invention wherein administering the capsule system containing insulin-producing islet cells to the subject allows the subject to maintain exogenous insulin independence for at least 10 days.

The invention also relates to a method of treating a large-mammal subject suffering from diabetes or related disorders with a cell therapy treatment that does not involve immunosuppression. The method comprises administering to the subject a cell therapy treatment of a composition containing insulin-producing islet cells that provides a sustained release of insulin for at least 30 days. The composition does not exhibit significant degradation during the sustained-release period.

The invention also relates to a method of treating a large-mammal subject suffering from diabetes or related disorders with a cell therapy treatment that does not involve immunosuppression, by administering to the subject a cell therapy treatment of a composition containing insulin-producing islet cells that provides a sustained release of insulin for at least 30 days, wherein the composition is a multi-membrane capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%. The capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams. This invention further relates to an invention wherein administering the capsule system containing insulin-producing islet cells to the subject allows the subject to maintain exogenous insulin independence for at least 10 days.

This invention also relates to a capsule containing a biological material that, when introduced into a large mammal having a functioning immune system, secretes a bioactive agent for at least 30 days without incurring significant degradation caused by immune attack from the immune system.

This invention also relates to a method of stabilizing the glucose level in a patient for at least 30 days, comprising administering to a patient suffering from diabetes or related disorders a cell therapy treatment of a composition containing insulin-producing islet cells. The cell therapy treatment is not administered in conjunction with an additional treatment involving immunosuppression.

This invention also relates to a method of stabilizing the glucose level in a patient for at least 30 days, comprising administering to a patient suffering from diabetes or related disorders a cell therapy treatment of a composition containing insulin-producing islet cells. The cell therapy treatment is not administered in conjunction with an additional treatment involving immunosuppression, wherein the composition is a multi-membrane capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%. The capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams. This invention further relates to an invention wherein administering the capsule system containing insulin-producing islet cells to the patient allows the patient to maintain exogenous insulin independence for at least 10 days.

### BRIEF SUMMARY OF THE DRAWINGS

FIG. 1: Biocompatibility of single-membrane capsules. Two single-membrane capsules prepared under identical formula and processing steps were photographed 30 days after being transplanted into intraperitoneally into a normal mouse (left) and a normal mongrel dog (right).

FIG. 2: Biocompatibility of multi-membrane capsules in a large animal. The omentum of normal dog is shown more than six months after treatment having capsules loosely adhered to the omentum.

FIG. 3: Permeability of capsule membrane. The chart illustrates normalized retention time as a function of pore size distribution of capsule membrane.

FIG. 4: Capsule mechanical stability. The chart illustrates the mechanical strength of capsules of two different polymer concentrations by plotting the rupture load versus the capsule membrane thickness and size.

FIG. 5: Capsule stability. The chart illustrates the mechanical strength of two capsules as a function of time with different chemical compositions and membrane thickness.

FIG. 6: Perifusion of encapsulated islets. The secretion level of insulin-releasing islets was assessed in a cell perifusion system. Free islets (not encapsulated), islets encapsulated in a single-membrane system (encapsulated islets), and islets encapsulated in a multi-membrane system (encapsulated with layer) were independently assessed.

FIG. 7: Insulin secretion by retrieved encapsulated islets. Islets encapsulated in a multi-membrane capsule retrieved after being transplanted in a dog at 100 days post transplantation were tested in a cell perifusion system.

FIG. 8: Blood glucose analysis of canine allotransplantation. The figure is an example of a canine model that has undergone a total pancreatectomy. The top panel illustrates the venous plasma glucose concentrations collected 12-18 hours following a meal. The lower panel illustrates the daily dosage of subcutaneous porcine insulin administered.

FIG. 9: Body weight analysis of canine allotransplantation. The top and bottom panels have been imported from FIG. 8. The middle panel shows the animal body weight monitored during the testing period.

FIG. 10: Fructosamine analysis of canine allotransplantation. The top and bottom panels have been imported from FIG. 8. The middle panel shows fructosamine measurements, an indicator of blood glucose level averaged over 2-3 weeks in diabetic subjects.

FIG. 11: Re-transplantation of encapsulated islets in canine. This chart illustrates an initial allotransplantation and re-transplantation on a canine of islets encapsulated in a multi-membrane system.

FIG. 12: Intravenous Glucose Tolerance Test (IVGTT). The chart evaluates intravenous dextrose (300 mg/kg) administration in a canine having previously received a transplantation of islets encapsulated in multi-membrane system.

FIG. 13: Pore size distribution (PSD) (FIG. 13A) and perifusion studies (FIG. 13B). FIG. 13A shows PSD of capsular membrane determined by exclusion of dextran solutes from capsules, where the x-axis represents pore size (R, in nm) and the y-axis (1−K_{sec}) represents an approximation of pore density of a given size pore. In FIG. 13A, the solid line presents the PSD of the current multi-membrane capsule design; the dotted line represents the PSD pattern of a membrane previously used to successfully reverse diabetes in small animal models. FIG. 13B shows insulin secretion by encapsulated canine islets in response to glucose challenge, where the x-axis represents a fraction collected from a cell perifusion system, and the y-axis represents insulin secretion (ng/ml) by that fraction, quantified by radioimmunoassay (not normalized for number of islets/cells per capsule).

FIG. 14: Data from a representative animal (#141) that received encapsulated islets intraperitoneally and maintained exogenous insulin independence for 214 days. FIG. 14A shows plasma glucose levels for animal #141 in mg/dl (y-axis) over approximately 250 days (x-axis), where samples for determination of venous plasma glucose were collected 6-18 hours after a meal. FIG. 14B shows insulin levels for exogenous insulin administered to animal #141 and circulating endogenous insulin levels in animal #141 over the study period. In FIG. 14B, exogenous pork insulin was administered on the days (x-axis) and in the amounts (Units, y-axis) shown by solid black histogram bars for regular insulin, and light gray shading for NPH. In FIG. 14B, circulating endogenous insulin (fasting plasma insulin concentration in μU/ml) assessed during the period of independence from exogenous insulin, is shown by dark gray shaded histogram bars.

FIG. 15: Data from a representative animal (#172) that received intraperitoneal administration of encapsulated islets on two occasions, with exogenous insulin independence maintained 66 days following the first administration and an additional 56 days following the second administration. FIG. 15A shows plasma glucose levels for animal #172 in mg/dl (y-axis) over approximately 220 days (x-axis), where samples for determination of venous plasma glucose were collected 6-18 hours after a meal. FIG. 15B shows insulin levels for exogenous insulin administered to animal #172 and circulating endogenous insulin levels in animal #172 over the study period. In FIG. 15B, exogenous insulin was administered to maintain glycemic control on the days (x-axis) and in the amounts (Units, y-axis) shown by solid black histogram bars (lower bars) for regular insulin, and light gray shading (upper bars) for NPH insulin. In FIG. 15B, circulating endogenous insulin (fasting plasma insulin concentration in μU/ml) assessed during the period of independence from exogenous insulin, is shown by dark gray shaded histogram bars.

FIG. 16: Intravenous Glucose Tolerance Test (IVGTT). IVGTT was performed on 4 animals within 6 weeks from transplantation. Dextrose (300 mg/Kg) was administered intravenously at T=0 min. Samples were collected from the jugular vein over 180 minutes (x-axis) for determination of plasma glucose (upper panel, in mg/dl) and insulin (lower panel, in μU/ml). Solid line with filled circle, control (n=5); dashed line with unfilled circle, dog #320; dashed line with unfilled triangle, dog #397; dashed line with open square, dog #259, solid line with solid square, dog #359.

FIG. 17: Images of capsules and encapsulated islets. FIG. 17A shows a digitized image (10× magnification) of “free floating” capsules retrieved from animal #141 after 214 days of exogenous insulin independence; capsule surfaces were clean with no sign of breaching; increased vascularized areas were present in conjunction with mildly adhered capsules, where approximately 50% of the encapsulated islets were mildly adhered to the omentum. FIG. 17B shows a digitized image of omental tissue and mildly adhered capsules retrieved from animal #397 after 102 days. FIG. 17C shows a digitized image (10× mag.) of an implanted capsule from animal #320, stained with hematoxylin and eosin, showing an intact capsule containing a morphologically normal islet. FIG. 17D shows a digitized image (40× mag.) of insulin immunostaining of an encapsulated islet from animal #108, where positive cytoplasmic staining for insulin can be seen.

### DETAILED DESCRIPTION OF THE INVENTION

Immunoisolation systems have been developed that allow for the effective and sustained encapsulation of biological material for cell therapy treatments. Any disease best treated by the release of a cellular product (hormone, protein, neurotransmitter, etc.) is a candidate for transplantation of immunoisolated cells. Potential cell types for immunoisolation include pancreatic islets, hepatocytes, neurons, parathyroid cells, and cells secreting clotting factors. When using encapsulating pancreatic islets in a cell therapy system, the system offers a surrogate bio-artificial pancreas and a functional treatment to a patient suffering from diabetes.

This invention relates to an immunoisolation system including a multi-membrane composition for encapsulating biological material, and methods of using the composition. In accordance with one aspect, an immunoisolation system has characteristics of a “gatekeeper” model, with a thin membrane having uniform pores that exclude high molecular weight immune system components such as IgM, while allowing low molecular weight oxygen, nutrients, and hormones to pass through without much impedance. It is recognized that many, if not most, immunoisolation systems include polymeric membranes (e.g., polymeric “gatekeeper” encapsulation systems) that may not have uniform pore sizes, and may include spectrum of pore sizes with a very long “tail” representing pore sizes outside the desired size range. In previous studies, polymeric gatekeeper encapsulation systems have been successful in small animal trials, and but was less than satisfactory in canine models and in human studies, sometimes requiring the use of immunomodulating and/or immunosuppressive agents in canine and human transplantation experiments (Soon-Shiong et al (1994) Lancet 343:950-951; Lanza et al. (1999) J Mol Med 77(1): 206-210; Calafiore (1999) Ann NY Acad Sci 875 219-232; Scharp et al. (2006) Am Diabetics [sic] Mtg Abstract 63-LB). In accordance with another aspect, an immunoisolation system has characteristics of a “barrier” model, wherein the capsule is a thick membrane with broad pore size distribution, where the membrane is not designed to keep all immune system components from entering the membrane, but small pores inside the thick membrane will prevent or delay most immune system components from entering the inner lumen wherein materials are encapsulated. Without wishing to be bound by this theory, a capsule having a capsule membrane is assumed to operate more like an immune system barrier than as a gatekeeper.

Without wishing to be bound by this theory, the invention relates to a multi-component and multi-membrane hybrid immunoisolation system that is understood to have desirable aspects of both the gatekeeper model and barrier model for immunoisolation systems. The invention relates to a multi-component, multi-membrane composition having aspects of gatekeeper and barrier function for immunoisolation. The invention relates to capsules according to the multi-membrane composition, wherein the capsules are capable of providing satisfactory immunoisolation after being administered to a subject.

The invention relates to a multi-component, multi-membrane composition that meets the “dichotomous” requirements for successful cell therapy without the need for immunosuppression, wherein the multi-membrane composition provides immunoisolation while also allowing release of agents from biological materials contained within the composition into a subject to which the composition has been administered, in order to effect the cell therapy treatment. The invention also relates to use of a capsule according to the multi-membrane composition, having biological material encapsulated within. The invention also relates to use of a capsule according to the multi-membrane composition having biological material encapsulated within, for cell therapy when the capsule is administered to a subject. The invention relates to use of a capsule according to the multi-membrane composition, having islet cells encapsulated within, for cell therapy without the use of immunosuppressive therapy.

This invention relates to a multi-membrane composition is a composition containing at least three membranes, wherein the composition is preferably either a capsule or a composition that has the ability to encapsulate biological material. It is understood that other systems in accordance with the multi-membrane composition may also work.

This invention relates to a multi-membrane composition comprising three membranes having different compositions and different properties, here referred to as an inner membrane, a middle membrane, and an outer membrane. This invention relates to a multi-membrane composition wherein membranes having different compositions and different properties, may be adjacent, interwoven, or a combination of adjacent and interwoven membranes. Without wishing to be bound by this theory, a multi-membrane composition with at least one membrane interwoven with another membrane may have certain desirable characteristics, for example, wherein a middle membrane is a thin interwoven that reinforces the inner membrane.

This invention relates to a multi-membrane composition for encapsulating biological material, comprising (a) an inner membrane that is biocompatible with the biological material and possesses sufficient mechanical strength to hold the biological material within the membrane and provide immunoprotection from antibodies in the immune system of a host; (b) a middle membrane that possesses sufficient chemical stability to reinforce the inner membrane from the chemicals in the host; and (c) an outer membrane that is biocompatible with the host and possesses sufficient mechanical strength to shield the inner and middle membranes from non-specific immune response systems in the immune system of the host. The middle membrane may, depending on the composition and structure, also bind the inner membrane with the outer membrane.

The inner membrane should be biocompatible with the biological material. That is, the biological material should not interact with the biological material in a manner that would kill or otherwise be detrimental to the biological material. The inner membrane should also possess sufficient mechanical strength to hold the biological material within the membrane and provide immunoprotection from antibodies in the immune system of a host.

The middle membrane possesses sufficient chemical stability to reinforce the inner membrane from the chemicals in the host. The chemical stability provided by the middle membrane also assists both the inner membrane and outer membrane in withstanding the effects of the chemicals in the host. Common chemicals in the host include sodium, calcium, magnesium, and potassium ions, as well as other chemicals in the bloodstream. The middle membrane is chemically stabile against those chemicals, which allows it to retard the deterioration of the membranes. This prolongs the life of the membranes and consequently the biological material that is being enclosed by the inner membrane. The middle membrane also binds the inner membrane with the outer membrane, preferably through affinity binding. Binding the membranes together in this manner provides a crosslinking effect that creates a tighter and more cohesive multi-membrane composition, and eliminates or reduces the possibility of membrane separation. The middle membrane can be interwoven with the inner membrane.

The outer membrane should be biocompatible with the host. Because the outer membrane is the portion of the multi-membrane composition that is contact with the host, it should be sufficiently biocompatible that the host does not treat the composition as a foreign object and reject it or attempt to destroy it. The term “biocompatible” as used in this context refers to the capability of the implanted composition and its contents to avoid detrimental effects of the host's various protective systems, such as the immune system or foreign body fibrotic response, and remain functional for a significant period of time. In addition, “biocompatible” also implies that no specific undesirable cytotoxic or systemic effects are caused by the composition and its contents such as would interfere with the desired immunoisolation functionality.

The outer membrane also should possess sufficient mechanical strength to shield the inner membranes from the non-specific innate immune system of the host. The innate immune system, which includes neutrophils, macrophages, dendritic cells, natural killer cells, and others, when activated, can attack the multi-membrane composition or capsule by engulfing it. It can also stimulate the activities of antibodies to attack the islets inside of the composition.

The combination of these features in the separate membranes allows the composition to jointly function in a manner not afforded by a single membrane. In particular, each membrane performs at least one function in a manner that allows the multi-membrane composition to meet the dichotomy goals of a large-animal transplantation. Each membrane is designed to allow optimal mass transport while maintaining islet health and functionality.

For instance, increasing the membrane pore sizes to improve mass transfer can jeopardize capsule stability. Likewise, increasing polymer concentration to improve capsule stability can decrease the mass transport. These dichotomies can lead to compromises on capsule design and performance. In the preferred system, no single membrane is required to compromise its design to meet the multi-faceted dichotomous goals. Each membrane is designed to perform only one or two specific tasks. Together, the multiple membranes meet most or all of the dichotomous goals of cellular transplants in a large animal model without the need for immunosuppression.

The membrane thickness of the inner membrane preferably ranges from about 5 to about 100 microns. More preferably, the membrane thickness ranges from about 10 to about 60 microns, and most preferably, the thickness ranges from about 20 to about 40 microns. Generally, the thicker the membrane, the more mechanical strength is provided. However, when a membrane becomes too thick, mass transport capabilities start to diminish.

The middle membrane typically has a thickness of less than about 5 microns (5 μm), preferably about 1-3 microns. The outer membrane typically has a thickness ranging from about 5 to about 500 microns, preferably ranging from about 100 to about 300 microns; however, a outer membrane thickness ranging from about 10 to about 30 microns is also suitable.

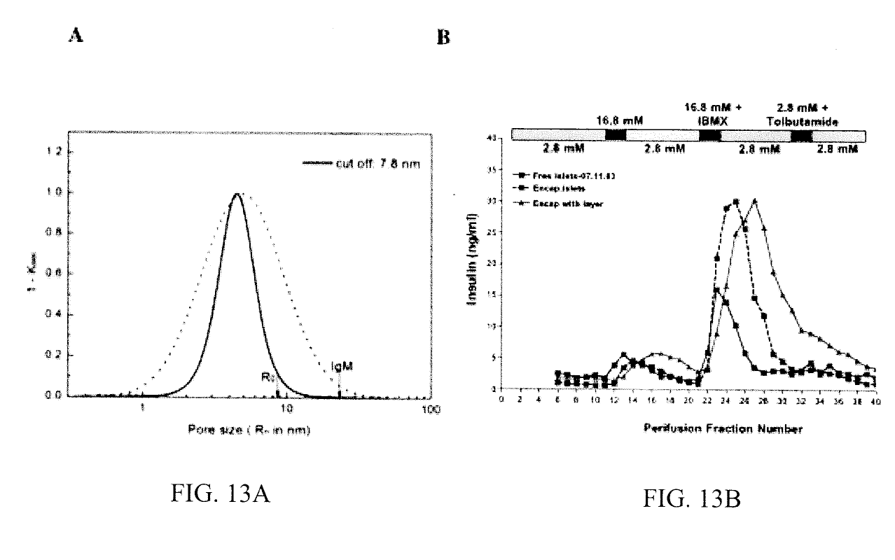

The multi-membrane composition has a porosity that is sufficiently large enough to allow for the release of bioactive agents from the biological material but sufficiently small enough to prevent the entry of antibodies from an immune system. There are known antibodies that destroy living cells that should, when possible, be prevented from entering the multi-membrane composition. For instance, the antibody IgM, which has a molecular weight of about 300 kilodaltons, can be particularly deadly when exposed to islet-containing capsules. In view of these known antibodies, the porosity cutoff (i.e., the considerable drop off of the number of pores larger than the cutoff size) of the multi-membrane composition should be less than 300 kilodaltons. Additionally, because membranes are often formulated as a random network system, the porosity cutoff is preferably below about 250 kilodaltons. This better assures that the designed membrane contains very few or no pores larger than 300 kilodaltons. In the non-limiting embodiment illustrated in FIG. 13A, the multi-membrane composition has a pore size distribution (PSD) clustered around a range of pore sizes sufficient to exclude IgM.

On the other hand, the porosity cutoff should be larger than about 50 kilodaltons to ensure that the biological material has the ability to be freely released from the multi-membrane composition. The porosity cutoff preferably ranges from about 50 kilodaltons to about 250 kilodaltons to permit the passage of molecules having a molecular weight less than about 50 kilodaltons while preventing the passage of molecules having a molecular weight greater than about 250 kilodaltons. More preferably, the porosity cutoff ranges from about 80 kilodaltons to about 150 kilodaltons.

In one embodiment of the invention, each membrane has a different porosity, with the inner membrane having a porosity cutoff ranging from about 50 to about 150 kilodaltons; the middle membrane having a porosity cutoff ranging from about 100 to about 200 kilodaltons; and the outer membrane having a porosity cutoff ranging from about 150 to about 250 kilodaltons. Having membranes of varying porosity assists, among other areas, in mass transport and immunoprotection.

This invention relates to a multi-membrane composition capable of encapsulating biological material, wherein the composition is a capsule system with at least three membranes with properties as described herein. In non-limiting embodiments, the average capsule pore size may be between about 10 nm to about 30 nm, where the average pore size may be about 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, or 29 nm. As illustrated in FIG. 13A, a capsule is understood to have a range of pore sizes, measured as the pore size distribution (PSD), from which an average capsule pore size may be calculated.

This invention also relates to a multi-membrane composition capable of encapsulating biological material, wherein the composition is a capsule system with at least three membranes with properties as described herein. In non-limiting embodiments, the capsule has a capsule diameter of between about 0.5 mm to about 1.1 mm, where the diameter may be about 0.6 mm, 0.65 mm, 0.7 mm, 0.75 mm, 0.8 mm, 0.85 mm, 0.9 mm, 0.95 mm, or 1.0 mm.

In non-limiting embodiments, the capsule has a wall thickness (i.e., the thickness of all three membranes that make up the capsule system) of between about 20 μm to about 70 μm, where the thickness may be about 25 μm, 30 μm, 35 μm, 40 μm, 45 μm, 50 μm, 55 μm, 60 μm, or 65 μm.

In non-limiting embodiments, the capsule has a mechanical strength greater than about 60 grams.

The biological material may be any material that is a capable of being encapsulated by a membrane. Typically, the biological material is a cell or group of cells that can provide a subject with some therapeutic result when introduced into the subject. Preferably, the biological material is selected from the group consisting of pancreatic islets, hepatocytes, choroid plexuses, neurons, parathyroid cells, and cells secreting clotting factors. Most preferably, the biological material is pancreatic islets or other insulin-producing islets capable of treating a patient suffering from diabetes.

The bioactive agent is any agent that can be released or secreted from the biological material. For example, pancreatic islets have the capability of secreting the bioactive agent insulin; choroid plexuses have the capability of secreting cerebral fluids; neurons have the capability of secreting agents such as dopamine that can effect the nervous system; and parathyroid cells have the capability of secreting agents that can effect metabolism of calcium and phosphorus in a subject. Preferably, the bioactive agent is insulin.

The host can include any subject that is in need or otherwise capable of receiving an encapsulated multi-membrane composition. While the host can include small mammals, such as rodents, the multi-membrane composition is particularly suitable for large mammals. Preferably, the host is a human.

While the multi-membrane composition should contain an inner, middle, and outer membrane, it may contain one or more additional membranes. Additional membranes may be desirable to provide better or more enhanced features to those provided by the three-membrane system. For instance, the additional membranes can, independently or jointly, provide additional immunoprotection, mechanical strength, chemical stability, and/or biocompatibility to the multi-membrane composition.

This invention also relates to a multi-membrane composition capable of encapsulating biological material, comprising (a) a membrane containing sodium alginate (SA), cellulose sulfate (CS), and a multi-component polycation; (b) a membrane containing a polycation; and (c) a membrane comprising a carbohydrate polymer having carboxylate or sulfate groups.

One membrane should contain sodium alginate (SA), cellulose sulfate (CS), and a multi-component polycation. The polycation is preferably contains a combination of poly(methylene-co-guanidine) (PMCG) and either calcium chloride, sodium chloride, or a combination thereof. This membrane may be the encapsulation system described in U.S. Pat. No. 5,997,900, herein incorporated by reference in its entirety.

A second membrane should contain a polycation. Preferably, the polycation is selected from the group consisting of poly-L-lysine, poly-D-lysine, poly-L,D-lysine, polyethylenimine, polyallylamine, poly-L-omithine, poly-D-omithine, poly-L,D-omithine, poly-L-aspartic acid, poly-D-aspartic acid, poly-L,D-aspartic acid, polyacrylic acid, poly-L-glutamic acid, poly-D-glutamic acid, poly-L,D-glutamic acid, succinylated poly-L-lysine, succinylated poly-D-lysine, succinylated poly-L,D-lysine, chitosan, polyacrylamide, poly(vinyl alcohol) and combinations thereof. More preferably, the polycation is selected from the group consisting of poly-L-lysine, poly-D-lysine, poly-L,D-lysine, poly-L-ornithine, poly-D-ornithine, poly-L,D-ornithine, chitosan, polyacrylamide, poly(vinyl alcohol), and combinations thereof. Most preferably, the polycation is poly-L-lysine.

The second membrane also preferably contains at least one compound selected from the group consisting of sodium alginate, cellulose sulfate, and poly(methylene-co-guanidine). More preferably, the second membrane contains a polycation and all three compounds. Most preferably, the second membrane contains poly-L-lysine, sodium alginate, cellulose sulfate, and poly(methylene-co-guanidine).

The third membrane contains a carbohydrate polymer having carboxylate or sulfate groups. The carbohydrate polymer preferably is selected from the group consisting of sodium carboxymethyl cellulose, low methoxy pectins, sodium alginate, potassium alginate, calcium alginate, tragacanth gum, sodium pectate, kappa carrageenans, and iota carrageenans. More preferably, the carbohydrate polymer is selected from the group consisting of sodium alginate, potassium alginate, and calcium alginate. Most preferably, the carbohydrate polymer is sodium alginate.

The third membrane also preferably contains an inorganic metal salt. Suitable metal salts include calcium chloride, magnesium sulfate, manganese sulfate, calcium acetate, calcium nitrate, ammonium chloride, sodium chloride, potassium chloride, choline chloride, strontium chloride, calcium gluconate, calcium sulfate, potassium sulfate, barium chloride, magnesium chloride, and combinations thereof. Preferably, the inorganic metal salt is selected from the group consisting of calcium chloride, ammonium chloride, sodium chloride, potassium chloride, calcium sulfate, and combinations thereof. Most preferably, the inorganic metal salt is calcium chloride.

In the multi-membrane composition, the first membrane is preferably the inner membrane, the second membrane is preferably an inner or middle membrane, and the third membrane is preferably the outer membrane. The multi-membrane composition may also contain one or more additional membranes.

Preferably, the multi-membrane composition is a five-component/three-membrane capsule system. The five components are sodium alginate (SA), cellulose sulfate (CS), poly(methylene-co-guanidine) (PMCG), calcium chloride (CaCl₂), and poly-L-Lysine (PLL). The inner membrane is the same PMCG-CS/CaCl₂-Alginate membrane successfully tested in small-animal models. This membrane is designed to provide a proper balance between immunoisolation and mass transport. The middle membrane is a preferably a thin interwoven PMCG-CS/PLL-Alginate membrane that reinforces the inner membrane. Strong ionic bonds, for example those present in the PMCG-CS/PLL-Alginate system, can assist in providing chemical stability. Additionally, having a thin membrane with a relatively large pore size can assist in allowing the membrane to not upset the balance between immunoisolation and mass transport of the inner membrane. The middle membrane can also provide impedance match for the inner and outer membranes by gradually increasing the PLL concentration of the middle membrane outwardly to bind the inner membrane with the outer membrane. An outer membrane of CaCl₂/Alginate shields the PMCG and PLL of the two inner membranes from the host immune system. This membrane improves the biocompatibility of the capsule and can also provide additional mechanical strength for stability as well as immune protection.

Preferably, the multi-membrane composition is a five-component/three-membrane capsule system with a capsule portion having an PMCG-CS/CaCl₂-SA inner layer stabilized with an interwoven membrane of PMCG-CS/PLL-SA, wherein the PMCG-CS/PLL-SA membrane is fused onto the PMCG-CS/CaCl₂-SA capsule, forming permanent bonds, and further including a third (outer) membrane of CaCl₂-SA. Without wishing to be limited by this theory, the PMCG-CS/CaCl₂-SA capsule (first, or inner, layer) is understood to function largely as a barrier membrane, the second (middle) PMCG-CS/PLL-SA membrane is understood to function as a gatekeeper membrane, and the third (outer) CaCl2-SA membrane encases the system and shields PMCG and PLL on the surface of the PMCG-CS/CaCl₂-SA capsule. Preferably, the PMCG-CS/CaCl₂-SA capsule includes SA at between about 0.6% to about 1.2%, CS at between about 0.6% to about 0.8%, CaCl₂ at between about 0.75% to about 1.0% CaCl₂, PMCG at about 1.2%, PLL at about 0.05%, and optionally includes an additional 0.05% PLL, and the CaCl₂-SA membrane includes CaCl₂ at between about 1.0% to about 1.5%, and SA at between about 0.9% to about 1.5%. Preferably, the multi-membrane system is a capsule system as disclosed in any of the embodiments in the Examples below.

This invention also relates to a method of treating a subject suffering from diabetes or related disorders, comprising administering to the subject sufficient amounts of a composition containing insulin-producing islet cells. The composition is a multi-membrane capsule comprising: (a) an inner membrane that is biocompatible with the biological material and possesses sufficient mechanical strength to hold the biological material within the membrane and provide immunoprotection from antibodies in the immune system of the subject; (b) a middle membrane that possesses sufficient chemical stability to reinforce the inner membrane from the chemicals in the subject; and (c) an outer membrane that is biocompatible with the host and possesses sufficient mechanical strength to shield the inner and middle membranes from non-specific immune response systems in the immune system of the subject.

Diabetes and related disorders include, but are not limited to, the following disorders: Type 1 diabetes, Type 2 diabetes, maturity-onset diabetes of the young (MODY), latent autoimmune diabetes adult (LADA), impaired glucose tolerance (IGT), impaired fasting glucose (IFG), gestational diabetes, and metabolic syndrome X. Preferably, the method is used to treat Type 1 diabetes or Type 2 diabetes.

The subject may be any animal that suffers from diabetes or related disorders. Preferably, the subject is a large mammal, such as a human.

Insulin-producing islet cells are preferably pancreatic islets, however, other cells capable of producing insulin are suitable. Porcine or human pancreatic islets are preferred, especially if the subject is a human.

This invention also relates to a method of treating a subject suffering from diabetes or related disorders, comprising administering to the patient sufficient amounts of a composition containing insulin-producing islet cells. The composition is a multi-membrane capsule comprising: (a) a membrane containing sodium alginate, cellulose sulfate, and a multi-component polycation; (b) a membrane containing a polycation; and (c) a membrane containing a carbohydrate polymer having carboxylate or sulfate groups. The polycation is in membrane (b) is selected from the group consisting of poly-L-lysine, poly-D-lysine, poly-L,D-lysine, polyethylenimine, polyallylamine, poly-L-omithine, poly-D-omithine, poly-L,D-omithine, poly-L-aspartic acid, poly-D-aspartic acid, poly-L,D-aspartic acid, polyacrylic acid, poly-L-glutamic acid, poly-D-glutamic acid, poly-L,D-glutamic acid, succinylated poly-L-lysine, succinylated poly-D-lysine, succinylated poly-L,D-lysine, chitosan, polyacrylamide, poly(vinyl alcohol), and combinations thereof. The multi-membrane composition may also contain one or more membranes in addition to the three discussed above.

This invention also relates to a method of treating a large-mammal subject suffering from diabetes or related disorders with a cell therapy treatment that does not involve immunosuppression. The method comprises administering to the subject a cell therapy treatment of a composition containing insulin-producing islet cells that provides a sustained release of insulin for at least 30 days. The composition does not exhibit significant degradation during the sustained-release period.

As known in the art, cell therapy is the transplantation of human or animal cells to replace or repair damaged or malfunctioning tissues, and/or cells. The types of cells that are administered correspond in some way with the organ or tissue in the patient that is failing. In the context of a subject suffering from diabetes or related disorders, cell therapy treatment involves the transplantation of insulin-producing cells that can replicate the function of pancreatic cells and release insulin into the subject upon the advent of certain conditions, namely an elevated glucose level in the subject.

A cell therapy treatment typically involves the introduction of either xenogenic (animal) cells (e.g., from sheep, cows, pigs, and sharks) or cell extracts from human tissue. The cells can be introduced through implantation, transplantation, injection or other means known in the art. Cells can be directly introduced into the host or introduced through cell encapsulation or special coatings on the cells designed to trick the immune system into recognizing the new cells as native to the host. Two general cell encapsulation methods have been used: microencapsulation and macroencapsulation. Typically, in microencapsulation, the cells are sequestered in a small permselective spherical container, whereas in macroencapsulation the cells are entrapped in a larger non-spherical membrane. Various polymeric materials have been used to form the membrane of the capsules in the encapsulation methods.

The cell therapy treatment preferably involves the transplantation of the encapsulated cells into the body cavity of the subject. This may be performed by creating a surgical opening in the body cavity and introducing the encapsulated cells into the body cavity through the opening. This may be accomplished through plausibly simple techniques, such as pouring the encapsulated cells into a funnel-type device that carries them through the opening and introduces them into the body cavity. Other techniques known in the art, such as hypodermic injections, may also be used.

Once inside the body cavity, the encapsulated cells are then able to freely move in the body cavity. Typically, the encapsulated cells will end up on the omentum of the subject. The omentum is a preferable place for the encapsulated cells because there is little danger of the cells interfering with the functions of the omentum. In contrast, if the encapsulated cells were to attach themselves to the outer walls of another organ, such as the liver or kidney, there is a chance that the encapsulated cells could disrupt the function of those organ, leading to other medical concerns.

The encapsulated cell therapy treatment is not administered with immunosuppressive agents designed to suppress a functioning immune system or otherwise prevent the immune system of the subject from rejecting the cell therapy treatment. Many cell therapy treatments require the use of immunosuppressive agent to ensure that the biological material being transplanted is not attacked and rejected by the immune system of the host. While immunosuppressive agents increase the chance that the host will accept the cell therapy treatment, it has been well documented that immunosuppressive drugs can cause deleterious effects to the host. In particular, immunosuppressive agents lower a subject's resistance to infection, make infections harder to treat, and increase the chance of uncontrolled bleeding. The drugs may also be harmful to the islets.

The term “sustained release,” as used herein, refers to the continual release of the biological agent from the biological material during instances when the release should take place. For instance, if the biological material is a pancreatic islet and the biological agent is insulin, the pancreatic islets should, after transplantation, continually release insulin into the host any time the pancreatic islets recognize that the glucose level of the host has reached a certain point. After the glucose level in the host has been maintained, the pancreatic islets will temporarily cease secreting additional insulin. However, when the glucose levels in the host again reach a point where insulin is needed, the temporarily-dormant pancreatic islets will again begin to secrete insulin. This type of continual release is an example of sustained release.

This invention relates to achieving and maintaining a condition wherein exogenous insulin is not required (i.e., exogenous insulin independence) after cell therapy treatment with encapsulated islet cells. It is understood that exogenous insulin independence follows sustained-release of insulin from encapsulated cells. This invention further relates to an invention wherein administering the capsule system containing insulin-producing islet cells to the subject allows the subject to maintain exogenous insulin independence for at least 10 days. In non-limiting embodiments of encapsulated islet cell therapy are disclosed in Example 2, and illustrated in Table 3, exogenous insulin independence was achieved and maintained for periods of between 65 to 214 days following a cell therapy treatment (intraperitoneal transplantation) with encapsulated islet cells, for a further period of between 50 to 129 days after a second cell therapy treatment with encapsulated islet cells.

The sustained-release period should last at least 30 days. Preferably, it lasts at least 60 days; more preferably, at least 120 days; even more preferably, at least 180 days, yet more preferably 200 days, and most preferably at least 220 days. The longer the composition is able to provide a sustained release of insulin, the longer the patient will be functioning on the cell therapy treatment alone without needing additional treatment. For instance, if the cell therapy treatment is able to last for at least 180 days, a patient will only need to receive a booster treatment approximately once every six months. This allows a diabetic patient a significantly increased amount of freedom to pursue daily activities without having to continually monitor their disorder and correct for high or low blood sugars and take insulin by injection or otherwise to counterbalance carbohydrate intake and regular and continual release of glucose into the bloodstream by the liver. This will also allow for overall greater glycemic control by reducing the occurrence of insulin shock or ketoacedosis as well as preventing or delaying the onset of diabetic related complications.

When a composition containing cells is effectively attacked by the immune system of a host, the immune system can damage or destroy the composition, causing significant degradation to the composition. There are two main avenues the immune system of a host can attack a foreign material, in this case a composition containing cells. First, the while blood cells in a host can either consume the composition containing the cells or adhere to the surface and suffocate the biological material inside. Second, the immune system of a host can generate specific antibodies that have the ability to penetrate the pores of a composition and attack the biological material inside. Either of these attacks will cause some form of degradation of the composition. However, if the composition contains sufficient biocompatibility, chemical stability, and mechanical strength the damage caused by the immune system and the degradation of the composition will be minimal. On the other hand, if the composition is not sufficiently biocompatible and chemically stable, and does not possess sufficient mechanical strength, the composition will be susceptible to attacks and the corresponding destruction caused by those attacks. Effective attacks will damage or destroy the biological material in the composition and leave the composition in a degraded state.

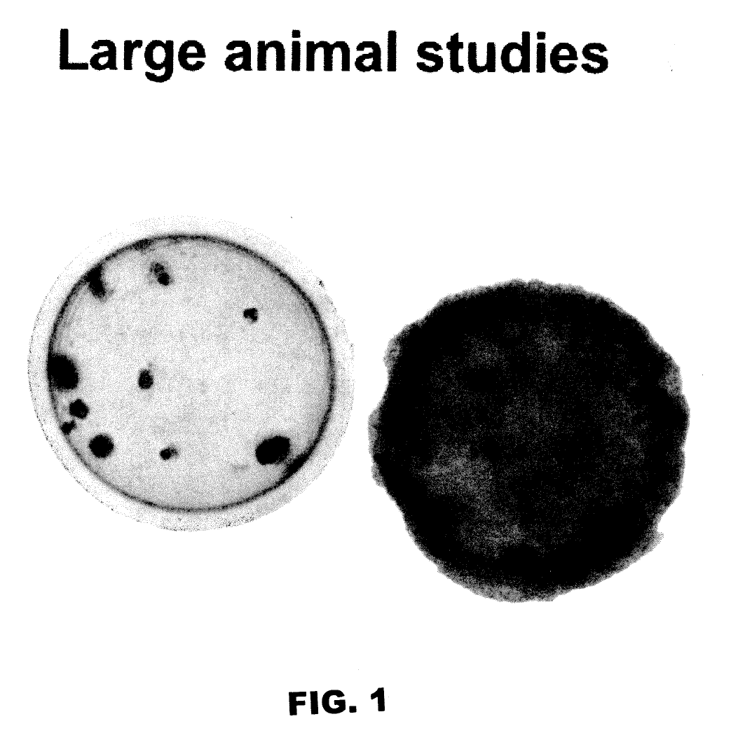

Conventional cell therapy systems, when introduced into large mammals, such as the canine model, were found to be stable for less than one month. An example of a conventionally produced encapsulated cell that experienced significant degradation in the canine model may be found in FIG. 1. FIG. 1 depicts two single-membrane capsules prepared under identical formula and processing steps were transplanted into intraperitoneally into a normal C57/B16 mouse (left) and a normal mongrel dog. Capsules were retrieved 30 days later and photographed. The rodent capsule shows no degradation while the canine capsule shows significant degradation due to breakage in the capsule and destruction of the biological material by the immune system of the host.

This invention also relates to a capsule containing a biological material that, when introduced into a large mammal having a functioning immune system, secretes a bioactive agent for at least 30 days without incurring significant degradation caused by immune attack from the immune system.

The term “capsule” refers to any type of encapsulation device used in an encapsulation system, including microencapsulation and macroencapsulation. Preferably, the capsule is a spherical capsule, such as those used in microencapsulation techniques. The capsule may be formed using special apparatuses and reactors, such as those described in U.S. Pat. Nos. 5,260,002 and 6,001,312, herein incorporated by reference in their entirety.

This invention also relates to a method of stabilizing the glucose level in a patient for at least 30 days, comprising administering to a patient suffering from diabetes or related disorders a cell therapy treatment of a composition containing insulin-producing islet cells. The cell therapy treatment is not administered in conjunction with an additional treatment involving immunosuppression.

As is well known in the art, patients suffering from diabetes or related disorders have glucose levels in their bodies that are not stabilized through a properly functioning pancreas. Stabilizing the glucose levels in diabetic patients or any other type of patient suffering from an instable glucose level, can be achieved through a cell therapy treatment of a composition containing insulin-producing islet cells. The cell therapy treatment can stabilize the glucose level for at least 30 days; preferably, at least 60 days; more preferably, at least 120 days; and most preferably, at least 180 days.

There are several known processes to prepare cells for encapsulation that may be utilized. One form involves extracting cells from the patient they are to be used on and then culturing them in a laboratory setting until they multiply to the level needed for transplant back into the patient. However, cell multiplicity has not yet been achieved for all types of cells, such as pancreatic cells. Another procedure uses freshly removed animal tissue, which has been processed and suspended in a saline solution. The preparation of fresh cells then may be either injected immediately into the patient, or preserved by being freeze-dried or deep-frozen in liquid nitrogen before being injected. Cells may be tested for pathogens, such as bacteria, viruses, or parasites, before use.

Porcine pancreatic islet cells may be harvested from the pancreases of pigs or piglets obtained from research laboratories or local slaughterhouses. Preferably, the pigs or piglets are specific pathogen free (SPF) animals that have been bred and monitored for the purpose of islet donation. Alternatively, neonatal islets, which contain nascent or not fully developed immune systems, fetal pig islets, which contain islets that are matured in the laboratory, or embryonic cells from stem cell research, which contain cells that may be regenerated in the laboratory, may also be used for supplying islets. Human islets that are donated from healthy patients theoretically represent a good source of islets and tend to have less immune problems. However, currently not enough human islets are donated per year, effectively preventing, as a practical measure, human islets from being used as a sole source of islets.

The invention also relates to methods of administering a multi-membrane composition comprising a capsule system containing insulin-producing islet cells, wherein the encapsulated islets survive and function to permit the withdrawal of daily exogenous insulin (i.e., achieve exogenous insulin independence) and provide fasting glycemic control for at least a defined period, without immunosuppression. Preferably, the multi-membrane composition is a five-component/three-membrane capsule system with an PMCG-CS/CaCl₂-SA inner layer stabilized with an interwoven membrane of PMCG-CS/PLL-SA, and covered with a third (outer) membrane of CaCl₂-SA as described herein. Preferably the encapsulated islets are transplanted into subjects have diabetic symptoms.

In non-limiting illustrative embodiments disclosed herein, in particular at Example 2, canine islets encapsulated in a PMCG-CS/CaCl₂-SA-PMCG-CS/PLL-SA-CaCl₂-SA capsule system were transplanted intraperitoneally into nine (9) pancreatectomized dogs (canine allotransplantation experiments), and the encapsulated canine islets functioned sufficiently to permit the withdrawal of daily exogenous insulin and provided fasting glycemic control without immunosuppression in all nine animals. Eight of the animals' transplanted encapsulated islets functioned for 64 to 106 days and one animal's islets functioned for 214 days.

In non-limiting embodiments of the invention, intraperitoneal dosages of at least about 50,000 encapsulated IEQ/kg body weight (IEQ, islet equivalents, is a measure of islet quantity by converting islet populations of differing size into measurements of islet volume) are administered. Preferably, between about 55,000 to about 87,000 IEQ/kg are administered, more preferably between about 73,000 to 87,000 IEQ/kg are administered. In the non-limiting embodiment disclosed in Example 2, administration of between about 73,000 to 87,000 IEQ/kg extended the length of time of exogenous insulin independent in six out of seven animals receiving the encapsulated islets. In the non-limiting embodiment disclosed in Example 2, a second transplant (i.e., re-transplantation of encapsulated islets) was effective in providing fasting glycemic control after the initial transplantation ran its course. The effectiveness of a second transplant was determined on the basis of observing a lack of measurable circulating insulin prior to transplant, then a return to pre-transplant exogenous insulin requirements at the conclusion of the experiment, and the absence of any identifiable pancreatic tissue at autopsy, which affirms that the animals lacked their own insulin throughout the course of the experiment.

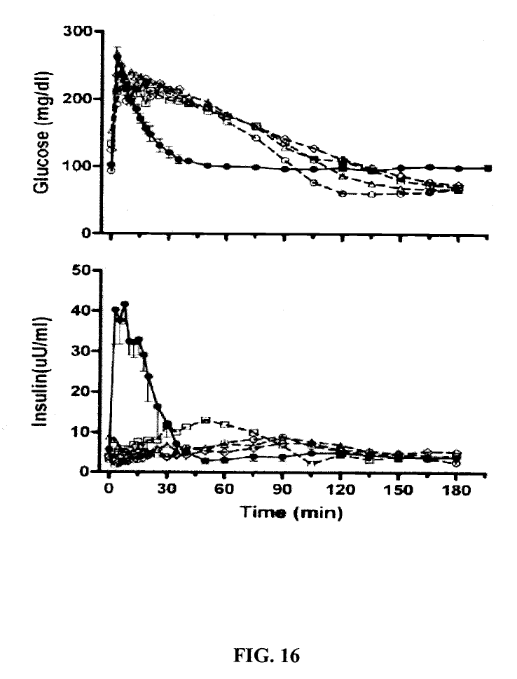

The invention also relates to methods of administering encapsulated islets as described herein, wherein encapsulated islets were responsive to glucose challenges (IVGTT and OGTT) with circulating glucose concentrations returning to baseline within 2 hours (Example 2, FIG. 16). The absence of an initial insulin response was observed in transplanted animals in both glucose challenge scenarios. Possible reasons for these results include impairments in transplanted islet physiology and/or transport dynamics related to the site of administration and/or diffusion controlled mass transport mechanism.

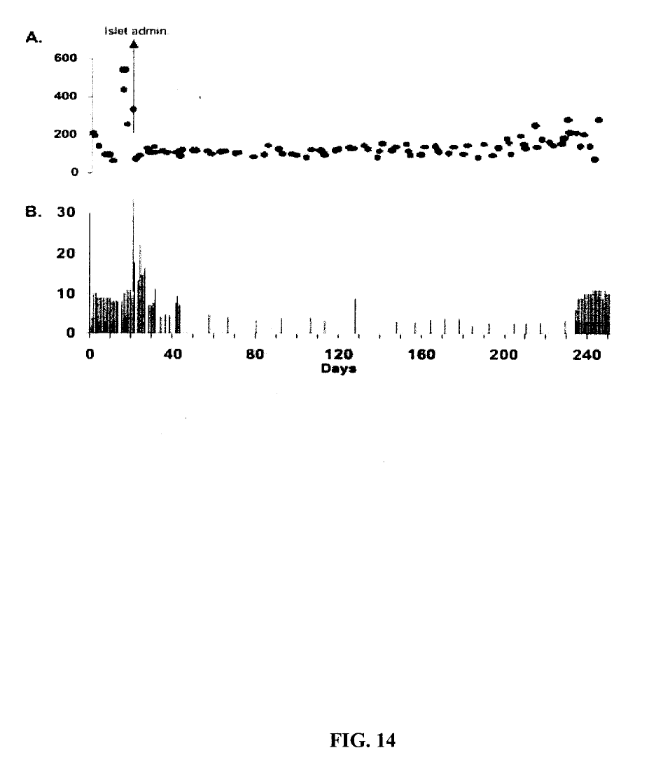

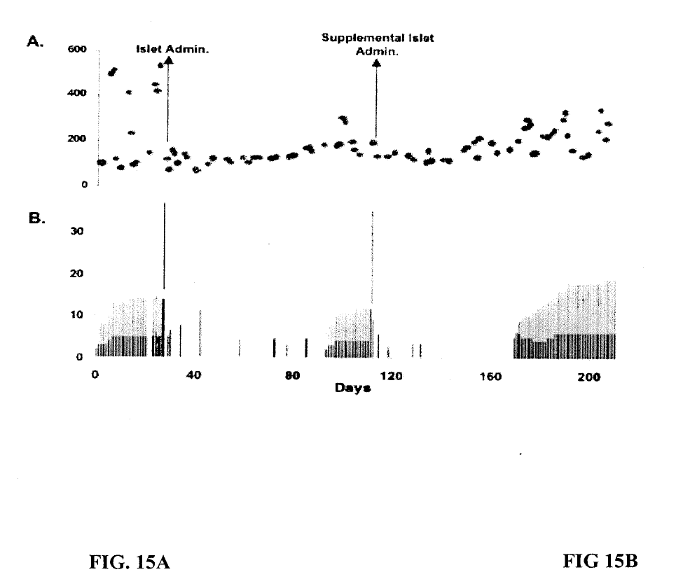

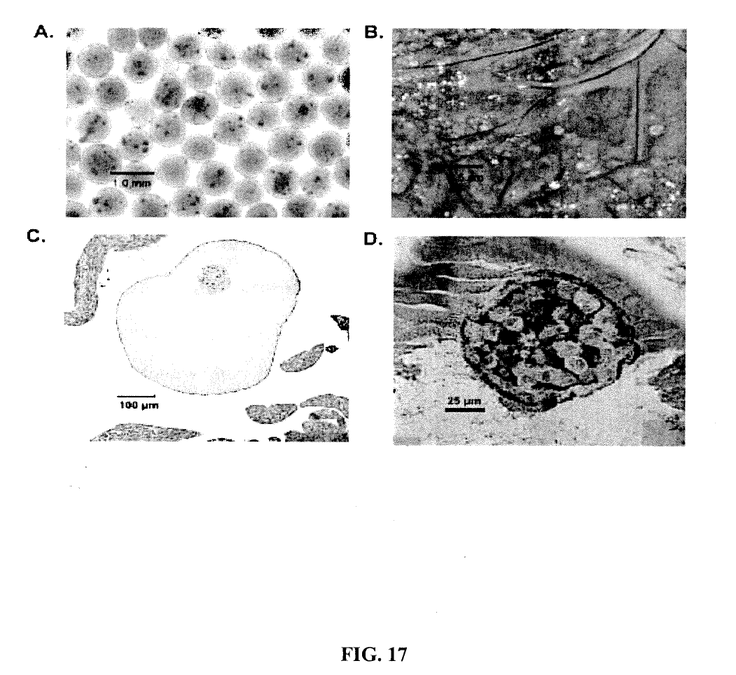

The invention also relates to methods of administering encapsulated islets as described herein, wherein capsules containing islets after transplantation can be found unattached and freely floating in the abdomen, or adhered to omental tissue after transplantation. In non-limiting illustrative embodiments, the majority of capsules adhered to omental tissue exhibited minimal pericapsular fibrosis (Example 2, FIG. 17A). In non-limiting illustrative embodiment, histological evaluations of capsules after transplantation showed that the majority of capsules were intact, and very few recovered capsules showed capsule breaching by inflammatory cells and fibroblasts/fibrocytes. In non-limiting illustrative embodiments, islets exhibiting normal morphology showed positive immunostaining for cytoplasmic insulin, thus indicating islet viability. In all embodiments in Example 2, which includes all transplantations including second transplant scenarios, circulating plasma insulin concentrations decreased during the first week of transplant suggesting early loss of function in a number of encapsulated islets (see, FIG. 14 and FIG. 15).

The invention also relates to methods of assessing the condition of a subject following administration of encapsulated islets as described herein. In certain embodiments disclosed in Example 2, the presence of islets exhibiting pathology, the presence of some degree of pericapsular fibrosis on capsules embedded within the omental tissue, the loss of islet viability in the early transplant period, and the absence of first phase insulin release were determined to all be potential indicators, if not contributors, for a need to return to exogenous insulin therapy.

The following examples are intended to illustrate the invention. These examples should not be used to limit the scope of the invention, which is defined by the claims.

### EXAMPLES

### Example 1

### Capsule Development and Preliminary Studies of In Vivo Function in Cell Therapy

Capsule Design: The following examples utilize a five-component/three-membrane capsule system. This system provides design flexibility to conduct systematic tradeoff studies to optimize capsule performance in large animals. The five components of the system are sodium alginate (SA), cellulose sulfate (CS), poly(methylene-co-guanidine) (PMCG), calcium chloride (CaCl₂), and poly-L-Lysine (PLL). The inner membrane is the PMCG-CS/CACL₂-Alginate (porosity of approximately 100 kDa, thickness of 20-40 micron); the middle membrane is a thin interwoven PMCG-CS/PLL-Alginate membrane (porosity of approximately 150 kDa, thickness of 1-3 micron); and the outer membrane is CaCl₂/Alginate (porosity of approximately 250 kDa, thickness of 100-300 micron).

Capsule Optimization: The following tests were performed to optimize the capsule. Because all the membranes should work together, it is difficult to predict how one membrane will affect another after the capsule has been fabricated. For instance, the process of forming the middle membrane can alter the performance of the inner membrane. Likewise, the process of forming the outer membrane can alter the performances of the middle and inner membranes. Additionally, advance characterization of each membrane individually does not predict how the multi-membrane capsules will function together inside transplantation hosts. For these reasons, capsule formation was treated as a total system with multiple parameters listed in the table below, with each membrane as a component. The desired function of each membrane was listed, and total performance of the system (capsule) was measured after fabrication.

TABLE 1
Reagents/Steps in Capsule Formation and Optimization

| | # | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Reagent/Step | PA 1 | PA 2 | PC 1 | RT 1 | PC 2 | RT 2 | PC 3 | RT 3 | PA 3 | PC 4 | RT 4 | PA 4 |

PA = polyanion;
RT = reaction time;
PC = polycation;

| | Membrane Formation Parameters |
|---|---|
| Capsule Design Parameter | (see #1-12 above) |
| Mass Transport (T) | 1, 2, 3, 4, 5, 6, 7, 8 |
| Immune Protection (P) | All |
| Biocompatibility | 9, 10, 11, 12 |
| Sphericity/Centering | 1, 2, 3, 4, |
| Stability (S) | 5, 6, 7, 8 |

Capsule performance: The multi-membrane composition was designed to be biocompatible, achieve effective mass transport, provide immune protection, provide mechanical strength to the biological material, and provide chemical stability.

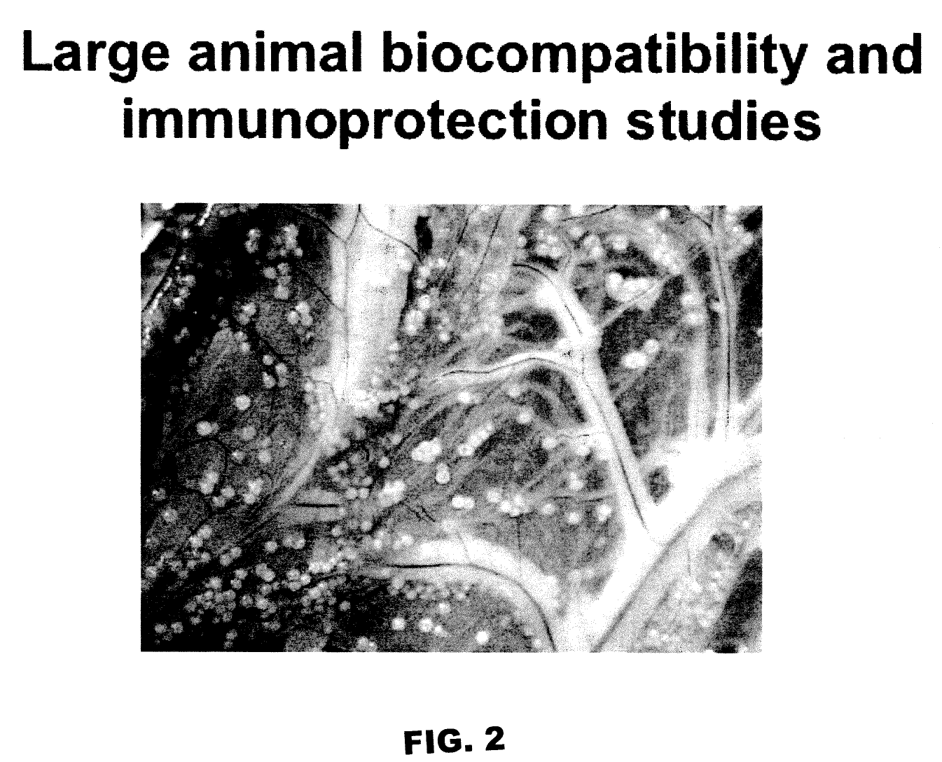

Biocompatibility: Biocompatibility of the capsules depends on shielding the immune-genesis components of the capsules from the transplantation host. Long-term biocompatibility of the capsule membrane was demonstrated when examination of encapsulated islets transplanted into a healthy dog for six and a half months revealed no complications. See FIG. 2.

FIG. 2 depicts the omentum of normal dog shown more than six months after treatment (dog received encapsulated islets on Feb. 14, 2001 and was sacrificed on Aug. 14, 2001). Before sacrifice, no complications were observed in the animal, and post sacrifice, no abnormalities were observed in or on the organs. The figure shows minimal inflammatory response and mild vascularization of the omentum. A few capsules (less than 1%), were observed to contain a scant amount of fibrin and rare mononuclear cells adherent to the surface. The surface of the vast majority of capsules retrieved from the dog were clean and transparent, and barely visible with the naked eye but readily apparent under microscope. Evidence of tissue reactivity has been minimal. There was no observed involvement of any other organ system in the splanchnic bed. The capsules loosely adhered to the omentum and were easily washed off, indicating that the capsules were anchored but not imbedded in the omentum. Capsule integrity was excellent with minimal capsule “breakage” observed. The retrieved encapsulated islets removed after six and a half months were still alive.

Mass Transport: Using interwoven pipes model, mass transport is proportional to R⁴/D, where R is average pore size, and D is the membrane thickness. See Wang T., “New Technologies for Bioartifical Organs,” Artif. Organs, 22, 1: p. 68-74 (1998), herein incorporated by reference in its entirety. The membrane pore size can be measured using the size exclusion chromatography method. See Brissova et al., “Control and measurement of permeability for design of microcapsule cell delivery system,” J. Biomed. Mat. Res., 39:61-70 (1998), herein incorporated by reference in its entirety.

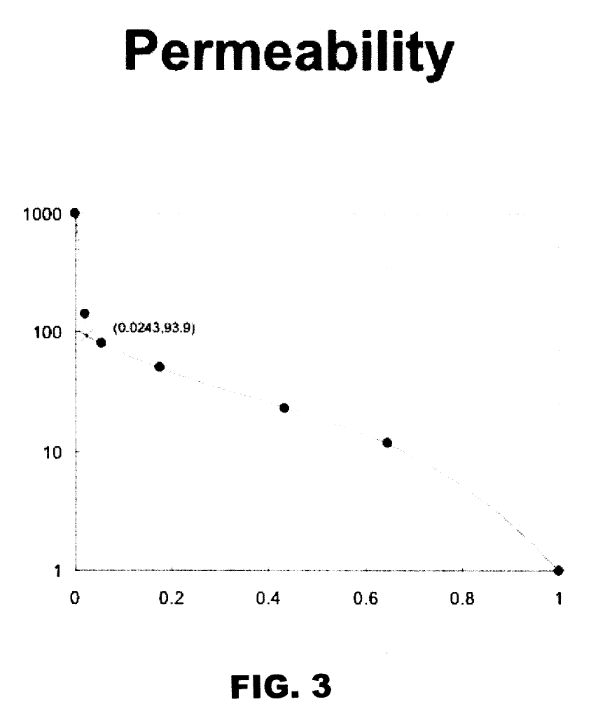

FIG. 3 demonstrates the pore size distribution of a capsule membrane with a cutoff of 80 KDa (about 12 nanometers in diameter). This pore size is large enough for the glucose and insulin to enter and exit, and small enough to keep the immune system from penetrate all the way to the core of the capsules where islets reside. The chart illustrates normalized retention time as a function of pore size distribution of capsule membrane. Pore size of the capsular membrane was determined by size exclusion chromatography (SEC) that measures the exclusion of dextran solutes from the column packed with microcapsules. The measured values of solute size exclusion coefficients (K_{SEC}) and known size of solute molecules allow the membrane pore size distribution and capsule permeability to be estimated.

Immune protection: Using random walk model, immune protection is proportional to D²/R², where D is the membrane thickness, and R is the average pore size. See Wang T., “New Technologies for Bioartifical Organs,” Artif. Organs, 22, 1: p. 68-74 (1998). In general, the immune protection goal is inversely proportional to the mass transport goals. However, their power dependences on membrane thickness and pore size are sufficiently different that it is possible to adjust the parameters to satisfy both goals simultaneously.

Mechanical strength: Mechanical strength of the capsules was measured by placing an increasing uniaxial load on the capsule until the capsule burst. The capsule mechanical strength, a function of membrane thicknesses, can be adjusted anywhere from a fraction of a gram to many tens of gram load to meet the transplantation goals without altering the permeability of the capsule.

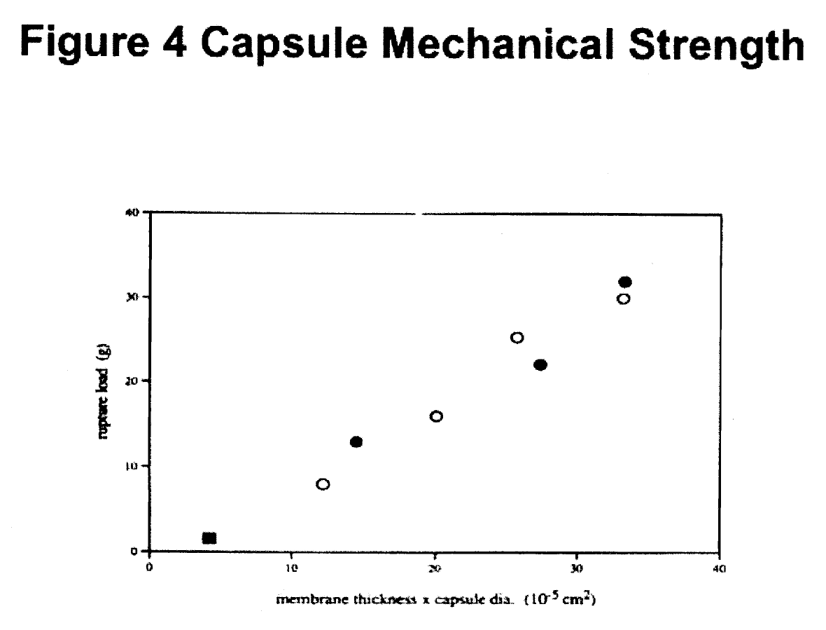

FIG. 4 illustrates the mechanical strength of capsules of two different polymer concentrations by plotting the rupture load versus the capsule membrane thickness and size. The slope of the curve represents the rupture stress and thereby indirectly the inherent strength of the capsular membrane. The chart measures mechanical burst strength of capsules by placing them on a uniaxial load. The solid circles represent 0.6-0.6 alginate-CS capsules, the open circles represent 0.9-0.9 alginate-CS capsules, and the solid square represents a PLL-alginate system. As can be seen in the chart, while certain polymers are stronger than others, it is generally observed that thicker membranes tend to be stronger membranes.

Stability: Stability of the capsules depends largely on the stability of chemical bonds and the membrane thickness. The intra peritonea fluid of a large animal such as dog can react chemically with the capsule membrane thus weaken the mechanical strength.

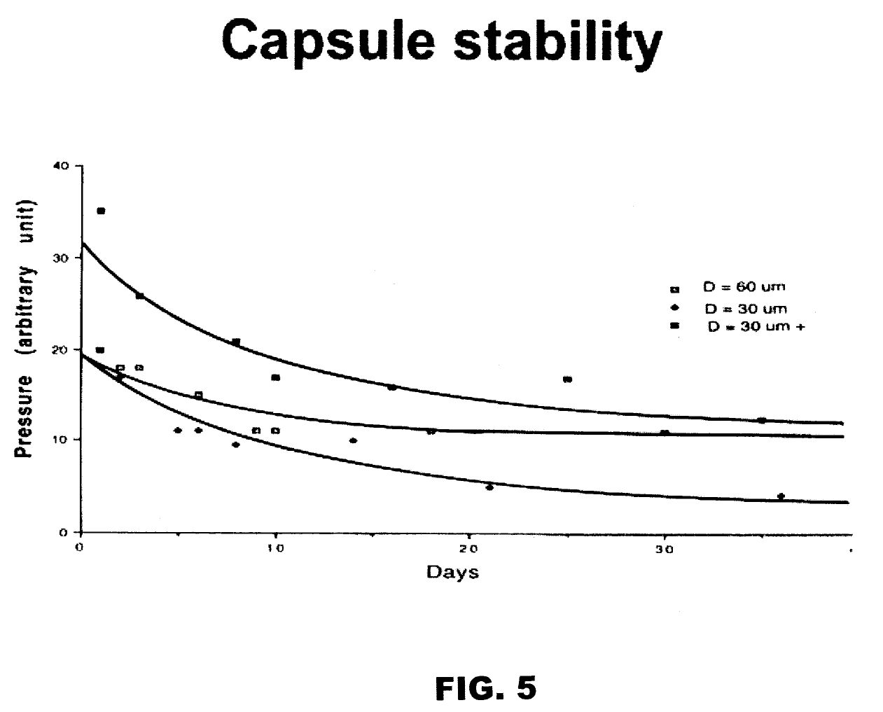

FIG. 5 illustrates the mechanical strength of two capsules with different chemical compositions and membrane thickness. The stability was experimentally determined by measuring the length of time for the capsules to loss its mechanical strength by a factor of 1/e incubated in dog serum at 40° C. It is believed that a properly designed capsule system can last years in a hostile environment of peritonea of a large animal. In FIG. 5, capsule mechanical strength was measured as a function of time as the capsules were incubated in dog serum at 40° C. The solid diamond represent 0.6-0.6 alginate-CS capsules, the solid squares represent 0.9-0.9 alginate-CS capsules, and the open squares represent 0.6-0.6 alginate-CS capsules. Stability is shown by the least amount of fluctuation over time. In the chart, the 0.6-0.6 alginate-CS capsules showed the least amount of fluctuation and would thus be considered the most stable capsules of the three tested.

The biocompatibility and functional capacity of the multi-membrane encapsulated islets has been studied in a pancreatectomized canine model. The animal's size and hence blood volume permits the daily evaluations of plasma glucose and insulin, clinical assessments of glucose tolerance and evaluations of biocompatibility and safety. In addition, the canine model is widely utilized model of human glucose homeostasis and diabetes. Total pancreatectomy in the canine results in complete absence of endogenous insulin and thus assessments of insulin concentration can be directly assessed to the function and responsiveness of the encapsulated islets.

Canine preparation: mongrel canines of either sex with a mean wt of 7.6 kg were studied. The animals were housed in a facility that met the American Association for the Accreditation of Animal care guidelines. All animal care procedures were reviewed and approved by Vanderbilt's Institutional Animal Care and Use Committee. Seventeen to twenty four days prior to encapsulated islet intraperitoneal administration, a total pancreatectomy was performed as described below. In the post-operative period animals are fed a standard diet of chow and canned diet (34% protein, 14.5% fat, 46% carbohydrate, and 5.5% fiber) based on dry weight. Exocrine pancreatic enzymes, lipase (70,000 U), amylase (210,000 U) and protease (210,000 U) were administered along with their meal in order to assist in food digestion and compensate for the absence of exocrine pancreatic function. Animals received daily insulin injections in adjusted dosages to maintain euglycemia at 12 hours post feeding without glycosuria during 24 hours. The insulin requirements generally range from 0.6-0.9 U/kg Regular Pork and 1.0-1.3 U/kg NPH Pork, q 24 hr.

Encapsulated Islet Administration: After pancreatectomy, daily insulin requirements were allowed to stabilize. Animals were fasted 12 hours and placed under general anesthesia using propofol (4.4 mg.kg, IV) and Isoflurane (2.0% with O₂, inhalation). A 1.5 cm midline laparotomy was performed and a 7.0 mm I.D. cannula is inserted into the peritoneal space. A funnel is connected to the free end of the cannula. Encapsulated islets suspended in modified Hanks solution containing canine albumin were administered into the abdominal space at room temperature. Total administered packed volume of capsules was 150-200 ml. The intraperitoneal cannula was immediately removed and the laparotomy incision closed. The animal was allowed to recover and immediately fed her/his daily ration. The ration was consumed within 2 hours from the time of the encapsulated islet administration. Six to eight hours post administration of food, blood was collected for the assessment of glycemic status and daily collections were performed thereafter for 3 days.

Daily and clinical assessments: Following the immediate post-administration period, animals were fed the standard daily rations and blood collections were performed on 2-3 day intervals for the determination of glucose and insulin. At the time of blood collections, animals were weighed and general physical conditions were assessed. An oral glucose tolerance test was performed at 2-4 weeks following encapsulated islet administration. Following an 18-hour fast, an 18-gauge angiocath was placed into either the left or right jugular vein for the collection of blood. Dextrose (0.7 gm/kg) was administered orally following the collection of a baseline blood sample. Blood samples were collected at 2.5-minute intervals for the first 20 minutes and 5 and 10-minute intervals thereafter for the 3-hour duration of the test. Plasma glucose levels were determined by the glucose oxidase method using a Beckman Glucose II analyzer (Beckman Instruments Palo Alto Calif.). Plasma insulin was determined by radioimmunoassay using a double-antibody system.

On the day of encapsulated islet administration, exogenous insulin is withheld and blood-glucose levels were monitored. No immune-suppressive drugs were administered to the animals.

Pancreatic islet isolation and evaluation: For the isolation of pancreatic islets, mongrel canines (20-28 kg body weight) were placed under general anesthesia following an 18-hour fast. A midline laparotomy was performed. The gastroduodenal, splenic and pancreaticoduodenal veins and arteries were isolated and a ligature was placed around each vessel. The main pancreatic duct was identified at the point of duodenal entry and dissected. A ligature was placed around the duct. An 18-gauge angiocath was inserted into the duct and the tip advanced 2-3 mm such that it remained in the main ductal architecture just prior to ductal branching in the pancreas. The catheter was sutured to the duct to secure its position. Immediately prior to harvest, the previously placed vascular ligatures were tightened and the animal was euthanized. The pancreas was transected from all peritoneal and vascular attachments and dissected from the duodenum. Once excised, the pancreas was immediately perfused with ice-cold University of Wisconsin (UW-D) perfusion solution via the previously placed ductal catheter.

A visual inspection was performed to ensure that the entire pancreas is perfused. The harvested glands were transported on ice to the laboratory where the UW-D solution was replaced by a solution of collagenase in UW-D (Crescent Chemical). The glands were then placed in a shaking water bath and digested at 40° C. for approximately 35 minutes. The dissociated tissue was filtered through a 400-μm mesh screen and washed several times with ice-cold media to remove and inactivate the collagenase. Based on density differences between islets and exocrine tissue, a discontinuous ficoll gradient was used to separate the islets and exocrine tissue. After density centrifugation, the islets were collected, washed, and transferred to tissue culture M199 media supplemented with 10% FBS (Fetal Bovine Serum) and antibiotics. During culture for 48-72 hours, isolated islets maintained their compact appearance and the capsule surface remained smooth.

Islet isolations were performed on 56 canine pancreases. A profile of the average isolation results per pancreas is shown below (islets fragments that are smaller than 50 μm are not quantified). In addition to the number of islets isolated, the quality of isolations was evaluated by determining the islet diameter, purity, islet viability, and islet function. Since the average islet diameter will vary, the isolation yield is normalized by computing the ratio of the average islet volume and the volume of a “standard” islet of 150 μm in diameter. The resulting value is referred to as the Equivalent Islet Number (EIN) and allows a yield-comparison for different isolations. Islet purity was determined from a sample that was stained with the islet-specific dye dithizone. The dye stains islets red but leaves exocrine tissue unstained. Most of the exocrine tissue dies during the first 24 hrs of culture, resulting in an increase in purity during culture to approximately 95%.

Islet viability is determined from a sample that was stained with a combination of Calcein AM (stains live cells fluorescent green) and Ethidium Bromide (stains the nuclei of dead cells fluorescent red). Viability is scored on a scale of 1 (all cells dead) to 4 (all cells alive). The average of five typical isolations is tabulated below.

| Islets per pancreas | 435 ± 38 K |
|---|---|
| Islet Diameter | 106 ± 3.8 μm |
| Islet Equivalent Number | 0.48 ± 0.04 |
| Purity | 87.3 ± 1.2% |
| Viability | 3.5 ± 0.1 |

Capsule formation and characterization: Capsules can be made with a droplet generator and a chemical reaction chamber, such as that described in U.S. Pat. Nos. 5,260,002 or 6,001,312, both of which are herein incorporated by reference in their entirety.

Another droplet generator system is a duo syringe system in which two or more syringes are connected in parallel and submerged in a temperature bath to keep the living cells healthy. The temperature bath containing the syringes may be an ice water bath having a temperature at about 4° C., which aids in keeping the cells in a dormant state. It has been found that islets, when in a dormant state, incur less damage during the transplantation process. This duo syringe system provides continuous operation by allowing for the refilling of one syringe while the experiment is ongoing with the other syringe. The syringes may also contain slow-turning propellers located inside the syringes that assist in maintaining islet density uniformity; i.e., more even distribution of the islets in the syringe.

The chemical reaction apparatus includes a multi-loop chamber reactor that is filled with solution, such as a cation solution. This cation solution bath is fed by a cation stream, which continuously replenishes the solution and carries away the anion drops being introduced into the chamber. Continuous SA/CS droplets can stream from the drop generator, with pancreatic islets enclosed, and enter the cation stream at a designated height and angle; so as to reduce or minimize islet decentering, drop deformation, and air bubble entrainment problems associated with impact. The droplets are then carried into the multi-loop reactor by the polycation stream. The reactor assists in controlling the time of complex formation as well as negating certain gravitational sedimentation effects. The capsules are carried into a second loop reactor with the same or different polycation solution for continuous operation. This facilitates tighter control of capsule diameter and sphericity as well as membrane thickness and uniformity.

Capsules may be produced with diameters ranging from about 0.5 mm to about 3.0 mm and membrane thicknesses ranging from about 0.006 mm to about 0.125 mm.

The mechanical strength of capsules may be measured by placing an increasing uniaxial load on the capsule until the capsule burst or totally compressed to a flat disc, as discussed previously and depicted in FIG. 4. The mechanical strength of the capsule, a function of membrane composition and thicknesses, can be adjusted anywhere from a fraction of a gram to many tens of grams load to meet the transplantation goals without significantly altering the permeability of the capsule.

A series of capsules having a range of permeability (porosity cutoff ranging from 40 kDa-230 kDa, based on dextran exclusion measurement) was developed and characterized. Capsule permeability can be measured by utilizing size exclusion chromatography (SEC) with dextran molecular weight standards. Measuring permeability and component concentration allows for the better control and manipulations of capsule permeability. The apparent pore size of the capsular membrane was determined by size exclusion chromatography (SEC) that measures the exclusion of dextran solutes from a column packed with microcapsules. By using neutral polysaccharide molecular weight standards, it is possible to evaluate the membrane properties under the conditions when solute diffusion is controlled only by its molecular dimension. Based on the measured values of solute size exclusion coefficients (K_{SEC}) and known size of solute molecules, the membrane pore size distribution (PSD) can be estimated.

### In Vivo Function of New Capsules

Encapsulated islets insulin secretion in response to stimuli: Following islet isolation, diameter, purity, and viability testing, the islets were cultured for 48-72 hours and encapsulated with a multi-membrane capsule. The insulin secretory capacity of the free islets and encapsulated islets was determined in a cell perifusion system, as described below. Insulin secretion by encapsulated islets was evaluated in a cell perifusion apparatus with a flow rate of 1 ml/minute with RPMI 1640 with 0.1% BSA as a perifusate. Encapsulated islets were perifused with 2 mM glucose for 30 minutes and the column flowthrough discarded. Three minute samples of perifusate were collected during a 30 minute perifusion of 2 mM glucose, a 30 minute perifusion of 20 mM glucose+0.045 mM IBMX (a nutrient), and a 60 minute perifusion of 2 mM glucose. Samples were assayed in duplicate for insulin using Coat-a-Count kits (Diagnostic Products Corporation, Los Angeles, Calif.) with a canine insulin standard. The amount of insulin secreted was normalized for the number of islets.

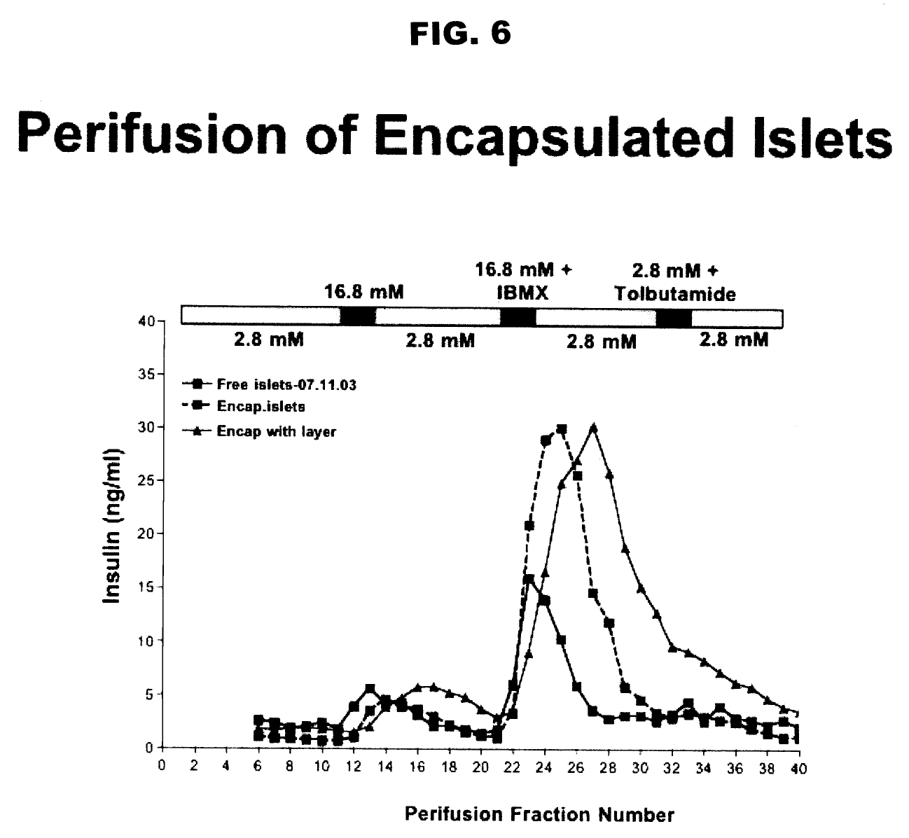

As assessed by the dynamic response to insulin secretagogues, insulin secretion by encapsulated islets had a similar profile as unencapsulated free islets with a slightly delay in insulin secretion. See FIG. 6. This delay in insulin secretion and the cessation of insulin secretion following removal of the stimulus reflects (a) the time for the secretagogue to enter the capsule and reach the islet and (b) the time for insulin to exit the capsule.

FIG. 6 depicts a cell perifusion system measuring the secretion level of insulin-releasing islets. Free islets (not encapsulated), islets encapsulated in a single-membrane system (encapsulated islets), and islets encapsulated in a multi-membrane system (encapsulated with layer) were independently assessed. Stimuli for insulin secretion are shown in the black bars at the top of the graph. Insulin in perifusion fractions collected every 3-minutes was quantified by radioimmunoassay. The number of islets was not normalized, so the focus of the chart should lie on the response time rather than the height of the graphs. The similarity of the response time in the three graphs with only minute delays suggests that the islets encapsulated in the multi-membrane system will function normally inside transplanted animals.

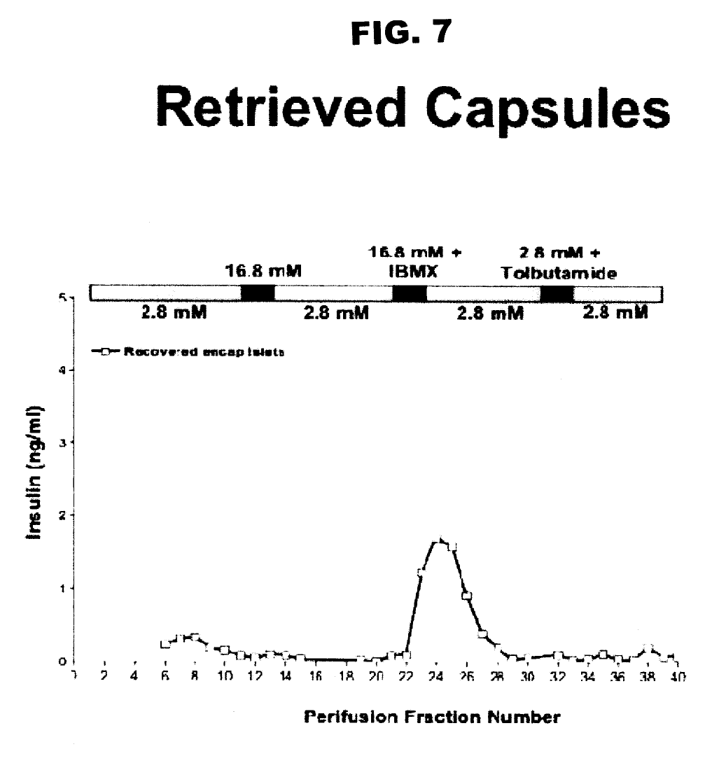

Encapsulated Islet Function and Safety: Using the total pancreatectomy dog model, the function and safety of the intra-peritoneally administered encapsulated canine islets (allograft) was assessed in 10 diabetic animals. The recurrence of diabetes, as determined by a glucose level of greater than 180 mg/dl for 4 consecutive days, occurred in dog 1 at approximately 100 days post transplantation. Encapsulated islets retrieved and tested in the cell perifusion system using the same stimuli as used in the previous transplantation shown in FIG. 6. See FIG. 7.

The chart in FIG. 7 indicates that the encapsulated islets are still viable as evidenced by the response to a high glucose plus IBMX, but have reduced insulin secretory capacity. These results suggest that the diabetes recurred because of inadequate islet mass and further suggest that this is due to reduced islet mass or function that is not the result of an allograft reaction.

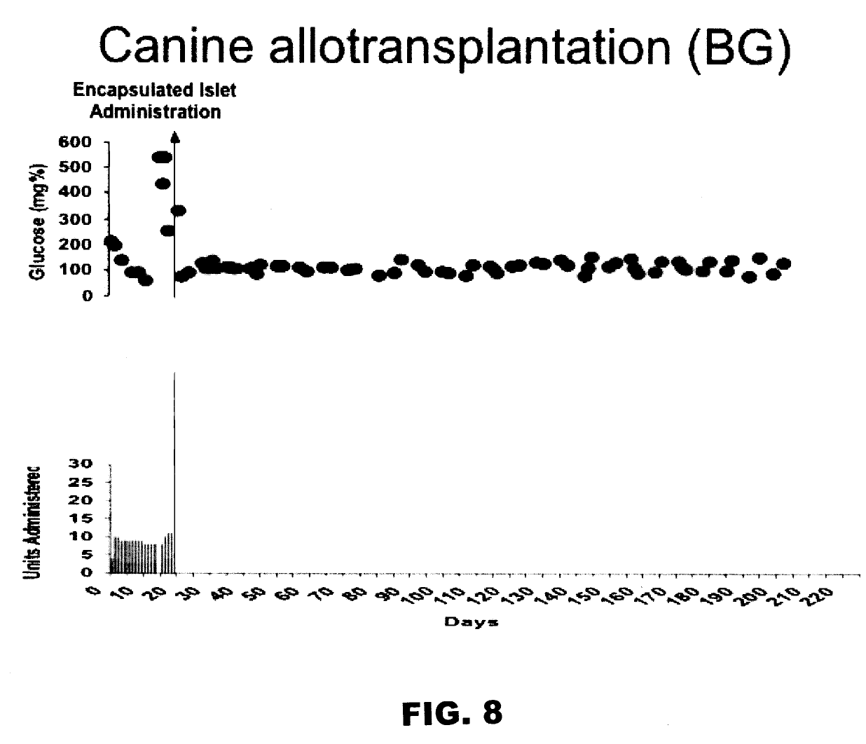

Fasting glucose concentrations, body weight, and fructosamine measurements of dog 10 are shown in FIGS. 8-10 as representative data. The retrieved capsules were clean and intact, suggesting that the longevity of the transplant is no longer limited by the capsule stability, but rather the loss of islet mass.

FIG. 8 depicts blood glucose analysis of canine allotransplantation. Transplantation of islets encapsulated in a multi-membrane system has demonstrated the efficacy in reversing diabetes in a canine model (dog no. 10) that has undergone a total pancreatectomy. The top panel illustrates the venous plasma glucose concentrations collected 12-18 hours following a meal. The lower panel illustrates the daily dosage of subcutaneous porcine insulin administered. The upper portion of bar in the lower panel indicates NPH insulin and the lower portion of bar indicates regular insulin. In days 18 and 19, treatments ceased to verify that the dog was diabetic. As seen in the top panel, glucose level rose dramatically when insulin treatments ceased. Insulin treatments resumed on day 20. On the morning of day 25, insulin treatments again ceased. In the afternoon of day 25, islets encapsulated in multi-membrane system were transplanted into the canine, as indicated by the vertical line. As illustrated in the top panel, glucose levels remained stabilized past day 200 at levels comparable or better than those observed during the period of insulin treatment. The bottom panel confirms that no additional insulin treatments were administered during this time period.

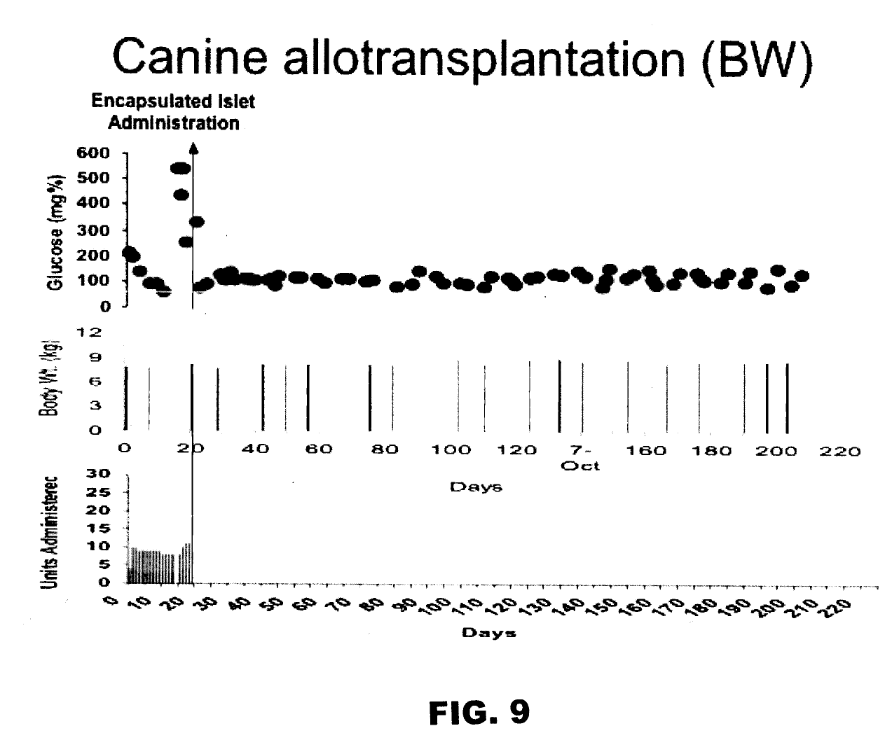

FIG. 9 depicts body weight analysis of canine allotransplantation. The top and bottom panels have been imported from FIG. 8. The middle panel shows the animal body weight monitored during the testing period. As can be seen in this chart, the body weight of the canine remained stable throughout the testing period.

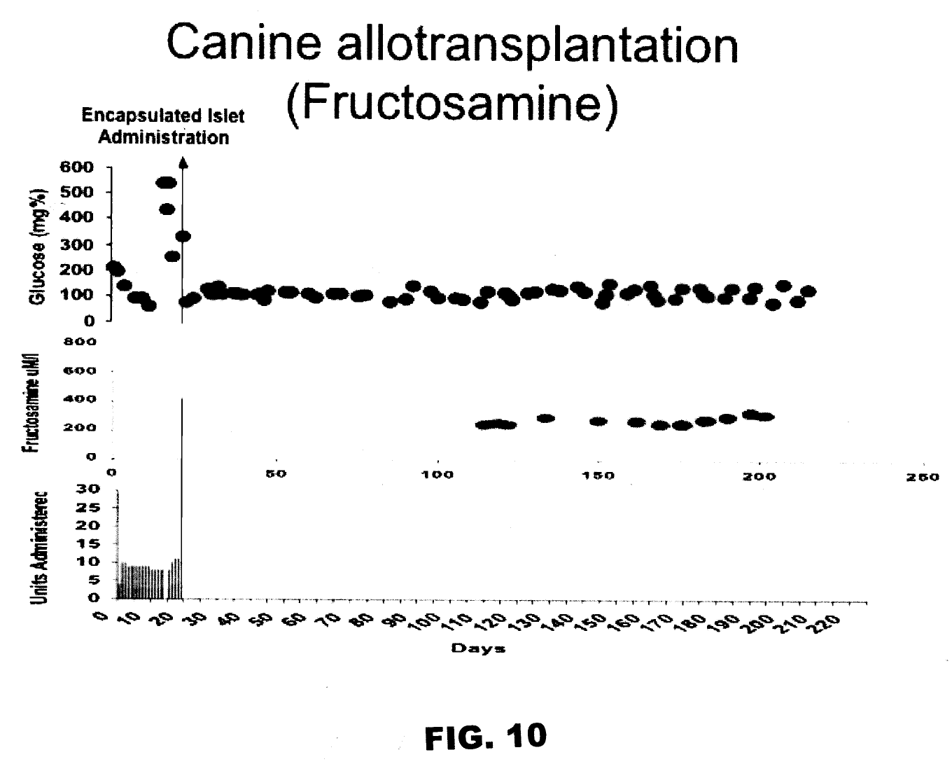

FIG. 10 depicts a fructosamine analysis of canine allotransplantation. The top and bottom panels have been imported from FIG. 8. The middle panel shows fructosamine measurements, an indicator of blood glucose level averaged over 2-3 weeks in diabetic subjects. A fructosamine level of 400 is roughly equivalent to an A1C measurement of 8.0, which is a similar indicator. The shaded area in the middle panel shows acceptable fructosamine levels. As can be seen in this chart, the fructosamine level on the tested days falls within the acceptable level. The tested fructosamine level is equivalent to an A1C level ranging from 6.0 (days 110-120) to 8.0 (days 195-200).

Re-transplantation: When fasting hyperglycemia recurs in animal, the transplant procedure may be repeated to maintain normoglycemia. For example, dog 7 received 40,000 EIN/kg, but was only able to maintain some semblance of glucose control for approximately 90 days. The dog was then given a second dosage of encapsulated islets of 63,000 EIN/kg total in two transplants (the transplants were administered a month apart due to the availability of the islets). The normoglycemia lasted approximately 110 days. These results are similar to those observed in the transplantation of dog 6 with 100,000 EIN/kg, and of comparable effectiveness in providing fasting glucose control.

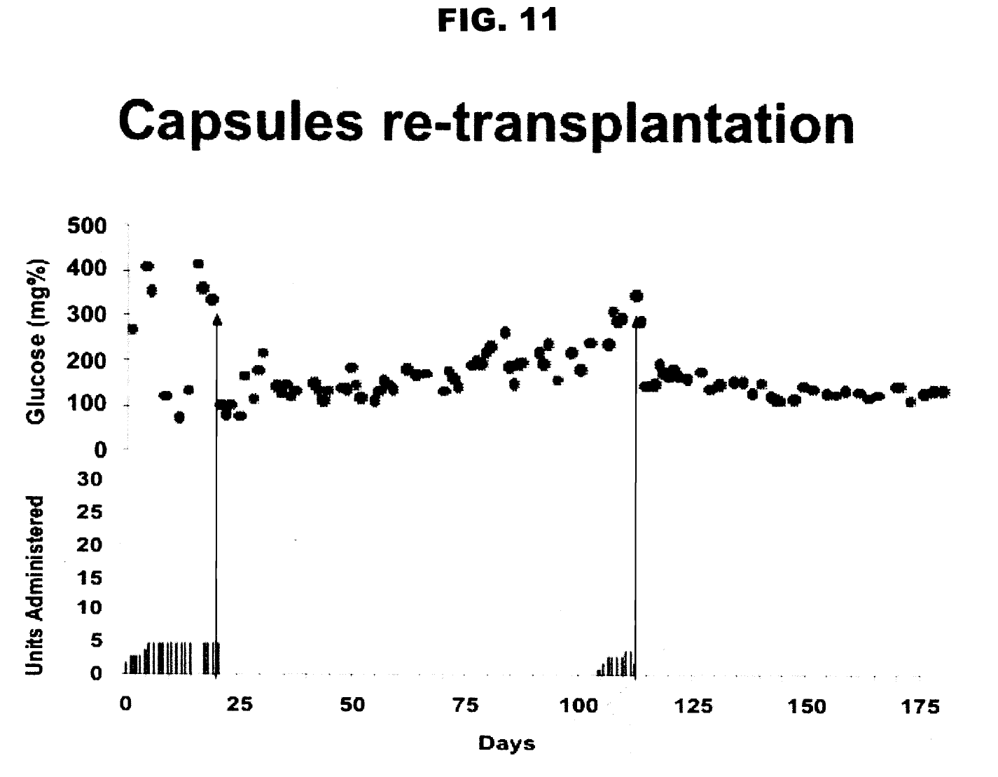

FIG. 11 shows the daily fasting blood glucose of dog 7 at 90-110 mg/dl without any supplemental insulin or immunosuppression. The vertical lines show the day of islet transplantation. The top panel shows data points that indicate the venous plasma glucose concentrations collected 12-18 hours following a meal. The lower panel indicates the daily dosage of subcutaneous pork insulin administered, with the upper portion of bar indicating NPH insulin, and the lower portion of bar indicating regular insulin. This figure illustrates the effectiveness of re-transplantation, as evidenced by the glucose levels stabilizing immediately after the second transplantation.

These results suggest additional transplants perform just as well if not better than initial transplantation. It is believed that the subsequent transplant performs better than the initial transplant because of the subject's ability to acclimate to the treatment and minimize vascularization. Re-transplantation provides improved glucose control and was well tolerated in the animal in terms of biocompatibility. Four successful re-transplantations have been performed on one subject; however, there is no practical limit to the number of re-transplantations that can be performed on a subject.

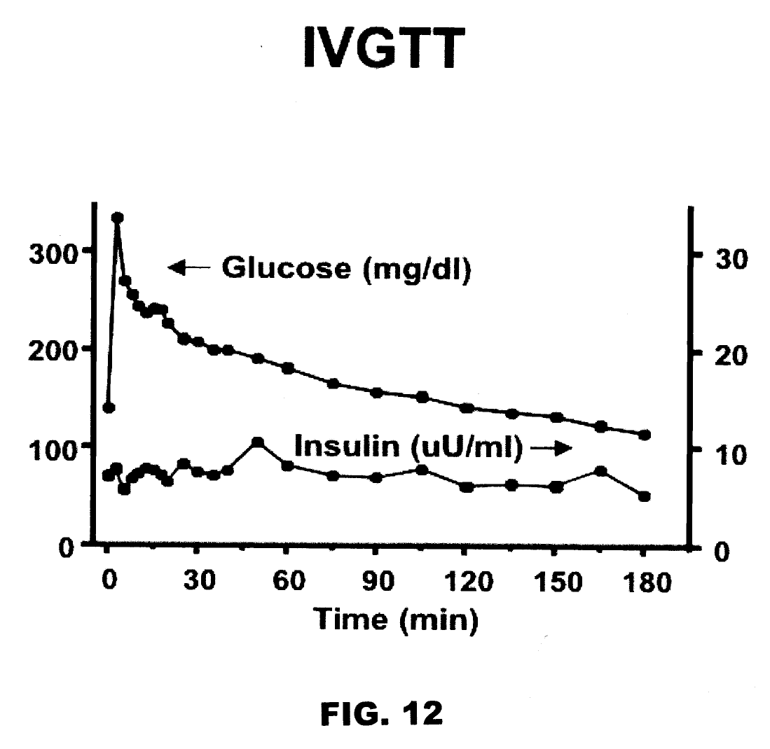

Intravenous glucose tolerance test (IVGTT): Intravenous glucose tolerance test (IVGTT) were performed on all animals to assess the in vivo function of encapsulated islets. FIG. 12 illustrates the IVGTT results of dog 5. Intravenous dextrose (300 mg/kg) was administered at T=0 in a canine having previously received a transplantation of islets encapsulated in multi-membrane system. Venous samples were collected from the jugular vein to determine plasma glucose and insulin.

The subject's blood-glucose level returned to normal at approximately 105 minutes, which is longer, but not unreasonably longer, than the 50-minute average exhibited by 6 control dogs. The rate of glucose clearing (The K value) was high, yet within normal range. Circulating insulin values for all the transplanted animals increased an average of 40% above basal in 75 minutes of the IVGTT and stayed at that level for the remainder of the test. Dogs with encapsulated islets did not demonstrate a first-phase insulin release that is often seen in the control animals. The lack of an insulin spike in response to glucose challenge (likely due to dilution effect of IP transplantation site) may have contributed to the islets gradually losing their ability to secrete sufficient insulin to maintain normoglycemia.

### Example 2

### Immunoisolation and Cell Therapy System Using Capsule System

The following example utilizes a five-component/three-membrane capsule system, assumed to include a random network system with a Gaussian distribution of nanopore sizes, to deliver insulin and protect cells from the host immune system. Administration of capsules produced as taught below, with islet cells encapsulated within, to totally pancreatectomized dogs (totally pancreatectomized canine allotransplantations), resulted in periods of exogenous insulin independence with no immunosuppressive therapy or any other modulating agents, i.e., successful cell therapy with successful immunoisolation.

Immunoisolation: Immunoisolation protection is calculated as the length of time “Γ” of a membrane, which keeps the immune system from breaching, expressed using Equation (1) below:

$$Γ \approx 3 \frac{{d}^{2}}{{R}^{2}} f τ \qquad ( 1 )$$

Where “R” is the nanopore radius, d²/R² is the number of random walk steps the immune system needs to take to breach the membrane thickness “d”, “f” is the frequency of collisions the immune system will have with nanopores between each step, and “τ” is the retention time of each collision.

Mass Transport: To maintain the islets' viability and function inside an immunoisolation capsule, nutrients and oxygen must be allowed to diffuse in, and the insulin out. Using a simplified interwoven pipe model (R>>r) and Einstein's relationship between diffusion coefficient and mobility, the effective mass transport “Q” passing through a polymer membrane pore can be approximated using Equation (2) as:

Q˜(R²T/dηr)ΔC (2)

Where “R” is the pore radius, “T” is the temperature, “η” is the viscosity, “r” is the radius of transporting particles, and “ΔC” is the concentration difference over membrane thickness “d.”

Equations 1 and 2 have an inverse relationship. What is good for immunoprotection is bad for mass transport and vice versa. A compromise in nanopore size and membrane thickness selections is needed to satisfy both immunoprotection and mass transport requirements.

Capsule Design: A new encapsulation system of sodium alginate (SA), CaCl2, polymethylene-co-guanidine (PMCG), cellulose sulfate (CS), and poly L-Lysine (PLL) was developed for large animal trials. This new system built on the PMCG-CS/CaCl2-SA barrier capsule which was successful in non-obese diabetic (NOD) mice trials, but not in large animals (Wang et al, (2000) “Bioartificial Pancreas: Principles of Tissue Engineering” 2nd ed., Chapter 36, 495-507, Academic Press; Wang (2002) Microencapsulation Methods: PMCG capsules: “Methods of Tissue Engineering” Academic Press pp. 841-852; Wang (1998) Artif Organs. 22, 1, 68-74; Wang et al. (1997) Nature-Biotechnology 15:358-362; Deng et al. (2007) J Fluid Mechanics (in press); Brissova et al. (1998) J Biomed Mat Res, 39, 61-70; Roth et al. (2006) Transplantation, 27; 81(8): 1185-90). To improve the performance, a thin interwoven PMCG-CS/PLL-SA gatekeeper membrane was fused onto the PMCG-CS/CaCl₂-SA capsule forming permanent bonds. This union was considered to improve the immunoprotection function without jeopardizing the mass transport function or vice versa. To shield the PMCG and PLL on the surface of the capsule, a third (outer) membrane of CaCl₂-SA was added to encase the system (Lanza et al. (1995) Transplantation 59, 1377-1384).

Capsule Development and Fabrication: The present multi-component and multi-membrane capsule design requires multiple processing steps. In each step, simultaneous chemical reactions take place, and each successive processing step impacts all previous steps. In addition, polymers vary from vendor to vendor and lot to lot, and can degrade with time. A variation in processing steps, or a minor change in polymers, can result in an unpredictable change in capsule performance. Therefore we did not use a “processing recipe” but instead utilized the capsule's diameter, membrane thickness, mechanical strength, and pore size(s) for processing control. Reagent concentration and reaction times for all nine capsules selected for transplantations are shown in Table 2 below. In general, the capsule fabrication process consists of three steps. Step 1 (reagent/time 1-6) forms the capsule; step 2 (reagent/time 7-8) improves capsule stability and ordering; and step 3 (reagent/time 9-11) improves capsule biocompatibility. Each step introduces additional chemical components and reaction times. Capsule characteristics were measured after every step.

TABLE 2
Membrane Processing Parameters

| Rgent | | | | | | | | | | | |
|---|---|---|---|---|---|---|---|---|---|---|---|
| Conc./ | | | | 4 | 5 | | | 8 | | | 11 |
| Reaction | 1 | 2 | 3 | Proc | PMCG/ | 6 | 7 | Proc | 9 | 10 | Proc |
| Time | SA | CS | CaCl2 | Time | PLL | Proc | PLL | Time | SA | CaCl2 | Time |
| Canine | 0.8% | 0.8% | 1.0% | 6 min | 1.2%/ | 0.16 min | | | 1.2% | 1.0% | 1.5% |
| #174 | | | | | 0.05% | | | | | | |
| Canine | 1.2% | 0.8% | 1.0% | 6 min | 1.2%/ | 0.5 min | 0.5% | 1 min | 1.5% | 1.5% | 5 min |
| #172 | | | | | 0.5% | | | | | | |
| Canine | 0.6% | 0.6% | 0.75% | 6 min | 1.2%/ | 0.25 min | | | 0.9% | 1.25% | 10 min |
| #320 | | | | | 0.5% | | | | | | |
| Canine | 0.8% | 0.8% | 1.0% | 6 min | 1.2%/ | 0.16 min | | | 1.2% | 1.0% | 8 min |
| #259 | | | | | 0.5% | | | | | | |
| Canine | 0.8% | 0.8% | 1.0% | 6 min | 1.2%/ | 0.16 min | 0.5% | 2 min | 1.5% | 1.5% | 10 min |
| #108 | | | | | 0.5% | | | | | | |
| Canine | 0.6% | 0.6% | 0.75% | 6 min | 1.2%/ | 0.25 min | | | 1.1% | 1.5% | 20 min |
| #359 | | | | | 0.5% | | | | | | |
| Canine | 0.8% | 0.8% | 0.75% | 6 min | 1.2%/ | 0.16 min | | | 1.2% | 1.1% | 10 min |
| #397 | | | | | 0.05% | | | | | | |
| Canine | 0.8% | 0.8% | 1.0% | 6 min | 1.2%/ | 0.16 min | | | 1.2% | 1.5% | 8 min |
| #249 | | | | | 0.5% | | | | | | |
| Canine | 1.2% | 0.8% | 1.0% | 6 min | 1.2%/ | 0.25 min | 0.05% | 1 min | 1.5% | 1.5% | 6 min |
| #141 | | | | | 0.05% | | | | | | |

When a new polymer or a new capsule design was being considered, capsule fabrication parameters were modified such that the final capsule characteristics achieved the desired result.

The apparatus for capsule fabrication has been previously reported (Anilkumar et al. (2001) J Biotechnol Bioeng, 75: 581; Lacik (et al.) 1998. J Biomed Mat Res 39: 52-60), and only a brief description will be given here. A multi-loop reactor filled with cation solution was continuously replenished by a cation stream to carry away the anion drops (SA and CS) being introduced into the chamber. The gelling steps were repeated for additional cation solutions till desirable capsule performance requirements were met. The capsules were coated with CA-SA to improve biocompatibility.

The apparent pore size of the capsular membrane was determined by size exclusion chromatography (SEC) that measures the exclusion of dextran solutes from the column packed with microcapsules. Using neutral polysaccharide molecular weight standards, it was possible to evaluate the membrane properties under the conditions when solute diffusion is controlled only by its molecular dimension. Measured values of solute size exclusion coefficients, and known size of solute molecules, were used to estimate the membrane pore size distribution (PSD); typical results in illustrated in FIG. 13A.

Using an apparatus developed in our laboratory, the mechanical strength of capsules was measured by placing an increasing uniaxial load on the capsule until the capsule burst. The capsule mechanical strength, a function of chemical bonds and membrane thicknesses, can be adjusted anywhere from a fraction of a gram to many tens of gram-load, to meet the transplantation requirement. Over two hundred capsule designs and variations in vitro were studied for this project. The capsules chosen for canine transplantation studies are summarized in the capsule parameter columns of Table 3 below.

Prior to animal studies, capsules were tested in a perifusion apparatus using RPMI 1640 medium with 0.1% BSA as a perifusate. The islets were equilibrated with perifusate containing 2 mM glucose at 37° C. On average, ˜100 encapsulated islets were loaded into each chamber of a multi-channel perifusion system. The flow rate in each channel was ˜1 ml/min. Samples of perifusate were collected over 3 min periods during a 30-min perifusion of 2 mM glucose, a 30-min perifusion of 2 mM glucose+0.045 mM IBMX, and a 60-min perifusion of 2 mM glucose. Samples were assayed in duplicates for insulin using Coat-a-Count Kits with an insulin standard. Typical results are illustrated in FIG. 13B.

Transplantation and Management: All methods and procedures regarding the use of animals were reviewed and approved by the Vanderbilt Institutional Animal Care and Use Committee.

Pancreatic Islet Isolation and Evaluation. Pancreases were surgically harvested from mongrel canines of either sex weighing 17-30 kg (avg. 22.4±0.4 kg). Briefly, the animals were anaesthetized and a surgical field created. The peritoneal cavity was opened by a long abdominal incision and the viscera exposed. The pancreas was partly mobilized, taking care not to sever any major blood vessels. The pancreatic duct was identified and cannulated at the duodenum. The animal was euthanized, and the excision of the pancreas completed. The gland was infused with cold modified University of Wisconsin (UW-D) organ preservation solution via the ductal cannula. The average harvested pancreas weight was 52±1.0 gm. The glands were transported on ice to the laboratory where the UW-D solution was replaced by a solution of collagenase in UW-D. The glands were then placed in a shaking water bath and digested at 40° C. for up to 30 minutes. The dissociated tissue was then filtered through a 400 μm screen and washed several times with ice-cold media to remove and inactivate the collagenase. Separation of islets and exocrine tissue was performed on a discontinuous ficoll gradient. After density centrifugation, the islets were collected, washed several times, and put in tissue culture in M199 media supplemented with 10% FBS and antibiotics. Islet purity was assessed with dithizone29 and was 90% or greater. Islet vital staining was performed with Calcein AM and Ethidium Bromide. Islet quantity was determined as islet equivalents (IEQ) using a formula to convert islet populations of differing size to islet volume. After losses during islet culture, handling, and encapsulation, the average yield was 80,000 IEQ of encapsulated islets per harvested pancreas.

Recipient preparation: Nine healthy mongrel canines of either sex, 6-11 kg (avg. 8.33±125 kg), were utilized as recipients. The canines underwent a total pancreatectomy 21-28 days prior to transplantation in order to render the animals totally insulin deficient. Exogenous purified pork insulin (regular and NPH) was administered to control hyperglycemia. Average exogenous pork insulin requirements were 0.6 and 1.2 U/kg regular and NPH insulin, respectively, during the pre-transplant period. Five to seven (5-7) days prior to transplantation, exogenous insulin was withheld for 36 hours in order to verify the completeness of the pancreatectomy and absence of circulating insulin. Pancreatic enzymes to aid in food digestion were administered along with the animals' daily food ration.

Transplantation. Five to seven (5-7) days prior to islet transplantation, insulin administration was withheld for 36 hours to verify the success of pancreatectomy. On the day of transplantation, general anesthesia was induced. A 2.5 cm midline incision was performed. Blunt dissection of midline adipose tissue and visualization of the abdominal contents were performed to ensure the intraperitoneal administration of encapsulated islets. Encapsulated islets were administered via a 6.0 fr. cuffed tube over 3-4 minutes, and then the incision was closed. Upon recovery from anesthesia (10-15 min), animals were provided their daily food ration, and exogenous insulin therapy was discontinued and not reinstated until the fasting glycemia was greater than 180 mg/dl for three consecutive days. No immunosuppressive or anti-inflammatory therapies were utilized.

Daily management and clinical assessments: The animals' general physical condition and dietary intake were monitored daily. Venous blood for the determination of glucose and insulin was collected at regular intervals. The animals had intravenous and/or oral glucose tolerance test (300 mg/kg, 0.7 gm/kg B.W., respectively) performed at monthly intervals. Normal (non-pancreatectomized) animals were utilized as normal controls for the IVGTT (n=5) and OGTT (n=5) assessments.

Histological Examination and Immunohistochemistry: At the conclusion of the experiments when exogenous insulin requirements returned to pre-transplant level, animals were euthanized, and omental tissue and encapsulated islets were retrieved and fixed in 10% neutral buffered formalin. Tissue sections were paraffin embedded, trimmed to 5 microns, and placed on charged slides followed by hematoxylin and eosin staining. (Richard Allan Scientific, Kalamazoo, Mich.)

Insulin Staining: Five-micron sections of paraffin-embedded tissue were placed on charged slides and deparaffinized. The sections were rehydrated and placed in heated citrate target retrieval solution (Labvision, Fremont, Calif.) for 20 minutes. Endogenous peroxidase was diminished with 0.3% hydrogen peroxide for 20 minutes followed by Ultra V block for 5 minutes (Labvision, Fremont, Calif.). Sectioned tissues were incubated with guinea pig anti-Insulin (Linco Research Inc, St. Charles, Mo.) diluted 1:5 K for 30 minutes. The Vectastain ABC Elite (Vector Laboratories, Burlingame, Calif.) system and DAB (DakoCytomation, Carpinteria, Calif.) were used to produce localized, visible staining. Slides were lightly counterstained with Mayer's hematoxylin, dehydrated, and cover slips applied.

Capsule Pore Size Optimization. Polymeric immunoisolation systems are random network systems. The nanopore size distribution of these membranes, as determined by size exclusion chromatography (SEC), consists of a spectrum of pore sizes with a long tail. As a result, both capsule design systems suffered limited success in large animal studies. Many of these large animal experiments were not reported or could not be reproduced. To overcome this, we have developed a new membrane design by fusing the PMCG-CS/PLL-SA membrane onto the PMCG-CS/CaCl₂-SA capsule, the strong ionic bonds of the PMCG-CS/PLL-SA system improve the capsule stability and reduce nanopore size distribution of the capsule by ˜50% as shown in FIG. 13A. This new hybrid capsule design operates like a barrier model with more uniform pore size distribution. This union, taking advantage of both the gatekeeper model and the barrier model, improves immunoprotection function without jeopardizing capsule mass transport or vice versa.

Capsule Perifusion Studies: Insulin secretion of encapsulated canine islets was evaluated in a cell perifusion apparatus. The insulin secretion by the encapsulated islets in response to glucose challenge as a function of fraction numbers (or time in minutes) is shown in FIG. 13B. The data shows a slight delay in response between the free islets and encapsulated islets. Encapsulated islets harvested 195 days post-administration are viable with signs of central necrosis (data not shown). This is likely due to the fact that nutrient transport by diffusion to the interior of encapsulated islets is inferior to the transport supported by islet vasculature. Similar conclusions have been reported by other groups with different capsule designs (Sun et al. (1996) J Clin Invest 98:1417-1422; Brissova et al. (2004) Diabetes, 53(5), 1318-25; De Vos et al. (1999) Diabetes, 48, 1381-1388; Duvivier-Kali et al. (2001) Diabetes 50: 1698-1705).

Transplantation studies: Five to seven (5-7) days prior to transplantation, exogenous insulin was withheld for 36 hours, and the measured circulating insulin levels were not detectable by radioimmunoassay, and hyperglycemia returned greater than 450 mg/dl. Within 6 hr of post-islet transplantation, exogenous insulin administration was not required. Animals exhibited average daily fasting glucose levels of 120-165 mg/dl and circulating insulin concentrations of 3-7 uU/ml at 6-18 hr post-meal between 64 and 214 days as shown in Table 3A below. Data from a representative animal (#141) is shown in FIG. 14. Three animals, #259, #108, and #172, received encapsulated islet supplementation (i.e., a second transplant) upon failure of initial transplant, and this second transplant was effective in providing extended control for 50, 129, and 56 days, respectively as shown in Table 3B below. FIG. 15 shows the course of events and data from a representative animal (#172) of the re-transplanted animals.

Exogenous insulin therapy was re-introduced when failure of the encapsulated islets to maintain fasting glycemia less than 180 mg/dl was evident for three consecutive days. When exogenous insulin requirements had stabilized at pre-transplant levels, animals were euthanized and an autopsy performed. All structures within the C-loop of the duodenum, including the duodenum, portal-hylar, and spleno-mesenteric regions, were closely examined and revealed no pancreatic tissue.

TABLE 3
Transplantation Parameters, Encapsulated Islet Longevity and Metabolic Parameters.

| | | Capsule Parameters | | | | | Days of | Avg. | Avg |
|---|---|---|---|---|---|---|---|---|---|
| | | | Pore | Thickness | Mech. | Post- | Exogenous | Glucose | Insulin |
| | B.W. | Diameter | Size | μm | Strength | encap | Insulin | Conc. | Concn. |
| Animal # | kg | mm | nm | (microns) | gm | IEQ/kg | Independence | mg/kg | μU/ml |

A. Data and capsule parameters for all animals after initial transplant

| | | | | | | | | | |
|---|---|---|---|---|---|---|---|---|---|
| 174 | 9.5 | 0.8 | 19 | 25 | ≧60 | 78,947 | 64 | 165 ± 10 | 3.7 ± 0.3 |
| 172 | 11.3 | 0.6 | 13 | 20 | ≧60 | 55,752 | 66 | 128 ± 7 | 5.7 ± 1.1 |
| 320 | 7.9 | 0.7 | 24 | 40 | ≧60 | 55,270 | 66 | 129 ± 9 | 3.1 ± 0.3 |
| 259 | 7.4 | 0.9 | 24 | 60 | ≧60 | 86,236 | 87 | 122 ± 10 | 5.2 ± 2.4 |
| 108 | 9.0 | 0.9 | 19 | 30 | ≧60 | 78,039 | 83 | 155 ± 12 | 4.7 ± 0.7 |
| 359 | 9.7 | 0.8 | 24 | 30 | ≧60 | 73,986 | 101 | 133 ± 6 | 3.8 ± 0.4 |
| 397 | 6.4 | 1.0 | 19 | 20 | ≧60 | 87,031 | 102 | 133 ± 4 | 5.4 ± 0.7 |
| 249 | 6.4 | 0.85 | 14 | 25 | ≧60 | 76,171 | 106 | 111 ± 4 | 4.6 ± 0.3 |
| 141 | 8.3 | 0.76 | 15 | 32 | ≧60 | 83,855 | 214 | 120 ± 4 | 7.2 ± 1.2 |

B. Data and capsule parameters for animals that received a second transplant

| | | | | | | | | | |
|---|---|---|---|---|---|---|---|---|---|
| 172 | 12 | 0.6 | 13 | 50 | ≧60 | 21,667 | 56 | 149 ± 11 | 5.2 ± 1.2 |
| 259 | 7.3 | 7.5 | 19 | 25 | ≧60 | 57,617 | 50 | 160 ± 14 | 5.4 ± 0.5 |
| 185 | 8.6 | 0.85 | 19 | 30 | ≧60 | 93,023 | 129 | 150 ± 6 | 5.4 ± 0.4 |

Glucose Challenges: IVGTTs and/or OGTTs were performed on all nine animals. FIG. 16 depicts the IVGTT results from control animals (n=5) and 4 transplanted animals. The glucose concentration in controls rose from baseline to 238 mg/dl by 5 minutes and returned to baseline by 60 minutes. In transplanted animals, the glucose levels rose from baseline to an average of 235±7 mg/dl by 5 minutes, returned to baseline by 95 minutes, and continued to fall to an average of 70±2 mg/dl at 180 minutes. Similarly, the insulin level in controls rose to 42±9 μU/ml by 7.5 minutes and returned to baseline by 35 minutes, and in transplanted animals rose to 8±2 μU/ml (60% above basal) by 50 minutes and returned to baseline by 135 minutes. Glucose and insulin results from animals that received an OGTT had similar response characteristics as IVGTT. In either the IVGTT or OGTT, animals with encapsulated islets did not demonstrate a first phase insulin release that was observed in the control animals.

Pathologic Evaluation: There were no complications associated with the encapsulated islets transplanted into the peritoneal cavity of healthy pancreatectomized dogs. There was evidence of omental neo-vascularization. Capsules were minimally attached but could be rinsed off the omental surface. Encapsulated islets that were mildly adhered to the omentum, or unattached and freely “floating” in the abdomen, were clean, and less than 1% of capsules had a slight amount of fibrin and rare mononuclear cells adhering to the capsule (FIG. 17A). There was no involvement of the encapsulated islets with any other organ system in the splanchnic bed.

Histological evaluation of omental sections with embedded capsules revealed some pericapsular fibrosis ranging from minimal to severe (one section) (FIG. 17B). Inflammatory cells (neutrophils, macrophages, and lymphocytes) were noted within the omentum and areas of fibrosis. Rarely, multinucleated giant cells were present. The inflammatory process was consistent with a foreign body reaction. Inflammatory cells and fibroblasts/fibrocytes were present within a small number of the capsules. Clusters of cells were embedded within the capsular matrix. These cells were consistent with the morphology of pancreatic islet cells and stained positive by insulin immunostaining (FIGS. 17C and 17D) The integrity of the cells was variable in the sections evaluated. Islet cells ranged from a normal appearance to exhibiting shrunken, pyknotic nuclei, loss of cellular margin, and mineralization.

Various modifications can be made to the embodiments without departing from the spirit and scope of the invention as defined in the appended claims.

1-22. (canceled)

23. A method of treating a subject suffering from diabetes or related disorders, comprising administering to the subject sufficient amounts of a composition containing insulin-producing islet cells, wherein the composition is a multi-membrane capsule comprising:
a. an inner membrane that is biocompatible with the biological material and possesses sufficient mechanical strength to hold the biological material within the membrane and provide immunoprotection from antibodies in the immune system of the subject;
b. a middle membrane that possesses sufficient chemical stability to reinforce the inner membrane from the chemicals in the subject; and
c. an outer membrane that is biocompatible with the host and possesses sufficient mechanical strength to shield the inner and middle membranes from the non-specific innate immune system of the subject.

24. The method of claim 23, wherein the diabetes or related disorders is a disorder selected from the group consisting of Type 1 diabetes, Type 2 diabetes, maturity-onset diabetes of the young (MODY), latent autoimmune diabetes adult (LADA), impaired glucose tolerance (IGT), impaired fasting glucose (IFG), gestational diabetes, and metabolic syndrome X.

25. The method of claim 23, wherein the subject is a large mammal.

26. The method of claim 25, wherein the large mammal is a human.

27. The method of claim 23, wherein the multi-membrane capsule has a porosity that is sufficiently large enough to allow for the release of insulin from the insulin-producing islet cells but sufficiently small enough to prevent the entry of antibodies from an immune system.

28. The method of claim 27, wherein the porosity cutoff ranges from about 50 kilodaltons to about 250 kilodaltons.

29. The method of claim 23, wherein each membrane performs at least one function in a manner that allows the multi-membrane composition to meet the dichotomy goals of a large-animal transplantation.

30. A method of treating a subject suffering from diabetes or related disorders, comprising administering to the subject sufficient amounts of a composition containing insulin-producing islet cells, wherein the composition is a multi-membrane capsule comprising:
a. a membrane comprising sodium alginate (SA), cellulose sulfate (CS), poly(methylene-co-guanidine)(PMCG), and calcium chloride (CaCl₂);
b. a membrane comprising a polycation selected from the group consisting of poly-L-lysine (PLL), poly-D-lysine, poly-L,D-lysine, polyethylenimine, polyallylamine, poly-L-ornithine, poly-D-ornithine, poly-L,D-ornithine, poly-L-aspartic acid, poly-D-aspartic acid, poly-L,D-aspartic acid, polyacrylic acid, poly-L-glutamic acid, poly-D-glutamic acid, poly-L,D-glutamic acid, succinylated poly-L-lysine, succinylated poly-D-lysine, succinylated poly-L,D-lysine, chitosan, polyacrylamide, poly(vinyl alcohol), and combinations thereof; and
c. a membrane comprising a carbohydrate polymer having carboxylate or sulfate groups.

31. The method of claim 30, wherein the polycation is selected from the group consisting of poly-L-lysine, poly-D-lysine, poly-L,D-lysine, poly-L-omithine, poly-D-ornithine, poly-L,D-ornithine, chitosan, polyacrylamide, poly(vinyl alcohol), and combinations thereof.

32. The method of claim 31, wherein the polycation is poly-L-lysine.

33. The method of claim 30, wherein the carbohydrate polymer is selected from the group consisting of sodium carboxymethyl cellulose, low methoxy pectins, sodium alginate, potassium alginate, calcium alginate, tragacanth gum, sodium pectate, kappa carrageenans, and iota carrageenans.

34. The method of claim 33, wherein carbohydrate polymer is selected from the group consisting of sodium alginate, potassium alginate, and calcium alginate.

35. The method of claim 30, wherein the membrane (b) further comprises at least one member from the group consisting of sodium alginate, cellulose sulfate, and poly(methylene-co-guanidine).

36. The method of claim 30, wherein the membrane (c) further comprises an inorganic metal salt selected from the group consisting of calcium chloride, magnesium sulfate, manganese sulfate, calcium acetate, calcium nitrate, ammonium chloride, sodium chloride, potassium chloride, choline chloride, strontium chloride, calcium gluconate, calcium sulfate, potassium sulfate, barium chloride, magnesium chloride, and combinations thereof.

37. The method of claim 36, wherein the inorganic metal salt is selected from the group consisting of calcium chloride, ammonium chloride, sodium chloride, potassium chloride, calcium sulfate, and combinations thereof.

38. The method of claim 30, further comprising one or more additional membranes.

39. A method of treating a large-mammal subject suffering from diabetes or related disorders with a cell therapy treatment that does not involve immunosuppression, the method comprising:
administering to the subject a cell therapy treatment of a composition containing insulin-producing islet cells that provides a sustained release of insulin for at least 30 days, wherein the composition does not exhibit significant degradation during the sustained-release period.

40. The method of claim 39, wherein the sustained-release period lasts for at least 60 days.

41. The method of claim 40, wherein the sustained-release period last for at least 120 days.

42. The method of claim 41, wherein the sustained-release period lasts for at least 180 days.

43. The method of claim 39, wherein the composition is a multi-membrane composition.

44. The method of claim 43, wherein the multi-membrane composition comprises at least three membranes, each of the membranes comprising at least one compound selected from the group consisting of sodium alginate (SA), cellulose sulfate (CS), poly(methylene-co-guanidine)(PMCG), calcium chloride (CaCl₂), and poly-L-lysine (PLL).

45. A capsule containing a biological material that, when introduced into a large mammal having a functioning immune system, secretes a bioactive agent for at least 30 days without incurring significant degradation caused by immune attack from the immune system.

46. The capsule of claim 45, wherein the biological agent is insulin.

47. The capsule of claim 45, wherein the large mammal is a human.

48. The capsule of claim 45, wherein the capsule secretes the bioactive agent for at least 60 days.

49. The capsule of claim 48, wherein the capsule secretes the bioactive agent for at least 120 days.

50. The capsule of claim 49, wherein the capsule secretes the bioactive agent for at least 180 days.

51. The capsule of claim 45, wherein the capsule is a multi-membrane capsule.

52. The capsule of claim 51, wherein the multi-membrane capsule comprises at least three membranes, each of the membranes comprising at least one compound selected from the group consisting of sodium alginate (SA), cellulose sulfate (CS), poly(methylene-co-guanidine) (PMCG), calcium chloride (CaCl₂), and poly-L-lysine (PLL).

53. A method of stabilizing the glucose level in a patient for at least 30 days, comprising administering to a patient suffering from diabetes or related disorders a cell therapy treatment of a composition containing insulin-producing islet cells, wherein the cell therapy treatment is not administered in conjunction with an additional treatment involving immunosuppression.

54. The method of claim 53, wherein the glucose level in stabilized for at least 60 days.

55. The method of claim 54, wherein the glucose level in stabilized for at least 120 days.

56. The method of claim 55, wherein the glucose level in stabilized for at least 180 days.

57. The method of claim 53, wherein the composition is a multi-membrane composition.

58. The method of claim 57, wherein the multi-membrane composition comprises at least three membranes, each of the membranes comprising at least one compound selected from the group consisting of sodium alginate (SA), cellulose sulfate (CS), poly(methylene-co-guanidine) (PMCG), calcium chloride (CaCl₂), and poly-L-lysine (PLL).

59-63. (canceled)

63. The method of claim 23, wherein the multi-membrane capsule containing insulin-producing islet cells is a capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%.

64. The method of claim 63, wherein the capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams.

65. The method of claim 64, wherein administering the capsule system containing insulin-producing islet cells to the subject allows the subject to maintain exogenous insulin independence for at least 10 days.

66. The method of claim 30, wherein the multi-membrane capsule containing insulin-producing islet cells is a capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%.

67. The method of claim 66, wherein the capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams.

68. The method of claim 67, wherein administering the capsule system containing insulin-producing islet cells to the subject allows the subject to maintain exogenous insulin independence for at least 10 days.

69. The method of claim 44, wherein the multi-membrane composition containing insulin-producing islet cells is a capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%.

70. The method of claim 69, wherein the capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams.

71. The method of claim 70, wherein administering the capsule system containing insulin-producing islet cells to the subject allows the subject to maintain exogenous insulin independence for at least 10 days.

72. The capsule of claim 52, wherein the multi-membrane capsule containing a biological material is a capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%.

73. The capsule of claim 72, wherein the capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams.

74. The method of claim 58, wherein the multi-membrane composition containing insulin-producing islet cells is a capsule system comprising three membranes wherein the inner membrane comprises a PMCG-CS/CaCl₂-SA capsule, the middle membrane comprises PMCG-CS/PLL-SA and the outer membrane comprises CaCl₂-SA, further wherein the PMCG-CS/PLL-SA middle membrane is an interwoven membrane fused onto the PMCG-CS/CaCl₂-SA capsule to form a structure comprising SA at between about 0.6% to about 1.2%, CS at between about 0.6% to about 0.8%, CaCl₂ at between about 0.75% to about 1.0% CaCl₂, PMCG at about 1.2%, PLL at about 0.05%, and wherein the CaCl₂-SA outer membrane comprises CaCl₂ at between about 1.0% to about 1.5%, and SA at between about 0.9% to about 1.5%.

75. The method of claim 74, wherein the capsule system has an average capsule pore size between about 10 nm to about 30 nm, a capsule diameter of between about 0.5 mm to about 1.1 mm, a wall thickness of between about 20 μm to about 70 μm, and a mechanical strength greater than about 60 grams.

76. The method of claim 75, wherein administering the capsule system containing insulin-producing islet cells to the patient allows the patient to maintain exogenous insulin independence for at least 10 days.
